(19) United States
(12) Patent Application Publication
(10) Pub. No.: US 2015/0037394 A1
(43) Pub. Date: Feb. 5, 2015

(54) DRUG DELIVERY FORMULATIONS

(71) Applicants: Daniel Guy Wood, Guildford (GB); Stuart Allen Jones, London (GB); Marc Barry Brown, Watford (GB)

(72) Inventors: Daniel Guy Wood, Guildford (GB); Stuart Allen Jones, London (GB); Marc Barry Brown, Watford (GB)

(73) Assignee: King's College London

(21) Appl. No.: 14/128,457

(22) PCT Filed: Jun. 22, 2012
(86) PCT No.: PCT/GB2012/051473

§ 371 Date: Apr. 29, 2014

(30) Foreign Application Priority Data
Jun. 22, 2011 (GB) 1110632.5

(51) Int. Cl. A61K 47/08; A61K 47/10; A61K 9/00

(52) U.S. Cl. CPC A61K 47/08; A61K 9/0014; A61K 47/10
USPC 424/449; 514/772

(57) ABSTRACT

Mixtures of C₁₋₁₂ compounds comprising at least one -Alk-O— group with thermogenic formulations, such as those comprising supercooled solutions of salts, are capable of substantial enhancement of transdermal drug delivery.

The present invention relates to methods for transdermal administration of drugs for both local and systemic delivery, and formulations for use in such methods.

The skin is the largest organ of the human body with an area of approximately 2 m² (Hadgraft, 2001). It is very easily accessible, has an excellent blood supply, and so appears to present an ideal location to administer therapeutic agents. However, the primary function of the skin is to act as a permoselective barrier, inhibiting the penetration of molecules and unwanted compounds into the body, thereby making drug delivery via the skin difficult.

Certain compounds penetrate skin to achieve therapeutic concentrations, either locally or systemically, via passive diffusion (Scheuplein et al., 1971). The passive transport of compounds into the skin usually occurs via three routes; transappendageal, e.g. through hair follicles, transcellular, i.e. through the cells, and paracellular, i.e. between the cells (Scheuplein, 1965). However, the majority of drug transport through the skin is thought to be paracellular (Choi et al., 2005).

For a compound to traverse the Stratum corneum (SC) efficiently, it must be relatively hydrophobic (log P of between 1 and 3) and have a low molecular weight (<500 Da) (Bos et al., 2000; Finnin et al., 1999). These stringent restrictions imposed by the SC limit the number of compounds that can pass into/through the skin and this has driven research into the development of safe and effective drug permeation enhancement strategies.

Strategies designed to overcome the barrier properties of the skin may be divided into passive methods, which rely on enhancing mass transport (e.g. chemical penetration enhancers, eutectics, supersaturated systems) and active methods, where the integrity of the skin is altered (e.g. microneedles, electrophoresis, iontophoresis, sonophoresis, phonophoresis, thermophoresis). Penetration enhancers work by increasing the solubility of a drug in the skin, enhancing partitioning and hence permeation (Moser et al., 2001). Although numerous penetration enhancers have been shown to be highly effective, an increase in drug permeation also often results in the permeation of other unwanted compounds, and can result in the accumulation and/or high systemic concentrations of potentially toxic agents. For example, azone, used commercially as a penetration enhancer, remains in the body for 5.5 days (Wiechers et al., 1988).

When a drug is saturated in a vehicle, the thermodynamic activity is described as being at unity i.e. equal to one. Drug thermodynamic activity is proportional to drug permeation rate. Accordingly, supersaturation systems increase the thermodynamic activity of a drug in a topical formulation above unity and, therefore, enhance permeation rate. However, supersaturated formulations are physically unstable and, over time, the drug in a supersaturated solution will precipitate, and this loss of drug from solution will eventually result in a return to a more stable saturated state. Accordingly, this enhancement approach has limited applicability in the clinical setting. Attempts have been made to improve the physical stability of supersaturated systems through the addition of antinucleant polymers (Iervolino et al., 2001), but this simply delays recrystallisation.

Drug permeation can also be increased by the application of a constant electrical current across the surface of the skin, an enhancement strategy known as electrophoresis (Wang et al., 2005). The electrical potential can be supplied by a wide range of devices but the most popular are patch based systems. Iontophoresis acts to improve drug delivery in three ways: electroporation, which relies on the formation of channels in the skin, due to the rearrangement of lipids, and through which the drug can travel; electroosmosis, whereby the migration of sodium ions across the skin carries water through with them as a result of the electrical current which, in turn, carries polar and neutral molecules through the skin; and electrorepulsion, wherein drugs with the same charge are forced through the skin (Chernomordik et al., 1991; Naik et al., 2000). Iontophoresis can increase the permeation of flux numerous compounds, but for the system to function effectively they must be charged. In addition, it is not possible to deliver large molecules using this technique (Prausnitz et al., 2004) and the patches tend to be bulky, visually unappealing and expensive.

Microneedles painlessly penetrate the Stratum corneum, with the intention that they do not penetrate the dermal layer where the nerve endings are located (Teo et al., 2006). A patch is often used to apply the microneedles and theoretically drug is transferred directly into the epidermis from the needle tips (Prausnitz, 2004). Typical needles on a patch are between 50-200 μm long and are commonly made from silicon (Teo et al., 2006). The main drawback associated with transdermal microneedle patches is their inability to achieve constant adherence to the skin and low loading capacity (Teo et at, 2006).

Thermophoresis, which requires the application of heat, is another method that can be used to increase the rate of drug permeation into and through the skin. The enhancement of drug delivery as a consequence of thermophoresis is attributed to heat increasing the drug diffusion kinetics, disruption of the lipid structure in the SC and/or an increase in local blood flow (Hull, 2002; Ogiso et al., 1998). The largest effect, ex vivo,is believed to be the effect of heat on the structural properties of the barrier. The application of heat to the surface of the skin causes structural changes by inducing lipid phase transitions within the SC, resulting in the barrier becoming more fluid and easier to penetrate. Several of these known low temperature (<50° C.) lipid transitions (Gay et al., 1994; Silva et al., 2006) are in physiological acceptable ranges.

Increasing the surface temperature of the skin has a major advantage over current enhancement methods in that it only temporarily disrupts the barrier properties of the skin and, therefore, does not induce the initiation of any subsequent repair (Tiwary et al., 2007). Although there has been relatively little research to determine the effect of heat on drug permeation compared to other enhancement strategies, previously published work has shown promise (Blank et al., 1967). For example, the effect of membrane temperature on the diffusion of three model penetrants, methyl paraben, butyl paraben, and caffeine, was shown to induce a maximum of a 1.6 fold enhancement in diffusion for every 7-8° C. increase in temperature (Akomeah et al., 2004). A 2-3 fold increase in nitroglycerin permeation has also been reported after 15 minutes exposure to an infrared bulb (Klemsdal et al., 1992), and a transdermal fentanyl patch containing an oxidative heating system increased drug delivery by more than 60% (Stanley et al., 2002).

Although this work provides evidence that heat can enhance percutaneous absorption, little research has focussed on clinically convenient methods or apparatus capable of utilising the theory. There are currently relatively few licensed heat generating products approved for human use across the world. The CHADD™ (Controlled Heat-Assisted Drug Delivery) patch is one of the few thermophoresis devices that have previously been developed specifically to enhance topical drug delivery. The heat from the patch can be generated for up to 12 hours and is produced via an oxidation reaction. Currently there is only one licensed product that utilises the CHADD™ technology, known as Synera™ in the US and Rapydan™ in the EU. While these heating patches can increase delivery, they also have several drawbacks. Skin irritation can occur as a result of the adhesive used to attach the patch. They cannot be applied to areas of the skin where hair is present, as the hairs cause the patch to loosen from the skin. Patches can be unsightly and large if the dose required to be delivered is high. They can also be uncomfortable, depending on where they are located on the body. In addition, and importantly, as the patches are activated by air, this poses manufacturing and packaging difficulties. Thus, while several products have previously been developed in an attempt to utilise this enhancement method, to date the commercial success of thermophoresis has been limited, as the devices designed to increase the surface temperature of skin are generally bulky, poorly adhesive, and often difficult to apply. As a result of the numerous inherent problems with current transdermal patch formulations, there remains a need for alternative and/or improved delivery systems either to enhance efficiency or consumer acceptability.

Supercooled or metastable salt solutions can produce heat as a result of a trigger condition. A supercooled solution is produced by heating a salt above its melting point e.g. sodium acetate or sodium thiosulphate, until it is fully melted, usually in the presence of a solvent, such as water. The mixture is then allowed to cool, and the salt effectively becomes supersaturated in the vehicle whilst remaining in solution. Exothermic crystallisation of the salt can be initiated by a trigger condition, such as a shear, or by the introduction of a nucleating agent (e.g. seed crystals) or fragments of a foreign material e.g. the flexion of a metal disc (as used in hand warmers).

It is possible to produce a variety of different temperature profiles using supercooled salt solutions, such as by altering the saturation level, the crystallisation rate, or the volume of supercooled solution used. Currently, supercooled solutions are commercially available as re-usable hand warmers and heat packs for sports injuries.

U.S. Pat. No. 4,077,390 describes reusable packs for warming hands, for example, containing a supercooled solution and means for activating the same. The heat pack is made by enclosing a supercoolable aqueous sodium acetate solution together with a metal activator strip in a sealed, flexible container.

U.S. Pat. No. 6,546,281 relates to the CHADD patch, such as referred to above, and discloses integrated apparatus for controlled heat aided dermal drug delivery. An integrated device comprises a drug formulation and a controlled heat aided drug delivery (CHADD) patch. A drug delivery compartment contains an active pharmaceutical ingredient (API). The CHADD heating system can reach temperatures of 39-43° C. for prolonged periods. The heat is generated from an oxidation reaction involving the components, activated carbon, iron powder, salt, water and fine wood powder. A selectively permeable membrane controls the oxidation reaction and the maximum heat and duration, as a consequence.

U.S. Pat. No. 4,230,105 discloses a method for the topical administration of systemically or locally active drugs, utilising a bandage containing one or more layers, including a drug and a heat generating substance. A patch type platform is structured as a heat generating area, or matrix, separated using a water impervious layer from a drug containing matrix/reservoir, and a pressure sensitive adhesive. The heat generating layer is an anhydrous inorganic hydrate salt dispersed throughout a matrix, wherein heat is generated upon contact with water.

Surprisingly, it has now been found that localised application of heat, such as may be generated by supercooled salt solutions, acts synergistically with lower alkanols, ethers, diethers, and related compounds, to enhance permeation of drugs into and across the skin,

Accordingly, in a first aspect, the present invention provides a thermogenic formulation for use in the transdermal administration of a drug, wherein said drug is present in said formulation or in a second formulation, one or more penetration enhancers being present in one or both formulations, and wherein at least one penetration enhancer is a C₁₋₁₂ compound comprising at least one -Alk-O— group, in which Alk is a C₁₋₆ alkylene and, when there is only one -Alk-O— group, then Alk may also represent a C₁₋₁₀ alkyl group.

The term ‘thermogenic’ is used herein to mean that a l ml quantity of a formulation is capable of generating sufficient heat to raise the temperature of the formulation to at least 40° C.

A thermogenic formulation may comprise any ingredients suitable to generate heat, and it is preferred that parameters associated with heat generation are controllable by the patient or skilled physician. Suitable thermogenic formulations may comprise supercooled salt solutions, which may be activated to yield heat by adding a crystal, for example. Other thermogenic formulations may comprise salts which yield heat of hydration, whereby the creation of the formulation is sufficient to trigger thermogenesis. Yet other thermogenic formulations may comprise monomers capable of yielding heat of polymerisation on the addition of a suitable catalyst, for example. Oxidation of iron when manipulated can also generate suitable heating profiles, as is well known in the art. In addition, heat can also be generated by the addition of water to propylene glycol and glycerine, for example.

It is preferred that the thermogenic formulation is capable of reaching temperatures of at least 40° C., and preferably no more than 50° C. Depending on the system used to generate the heat, the skilled person is capable of selecting the quantity of heat generating ingredients and the volume of solvent in order to achieve at least 40° C. and keep the maximum temperature below 50° C. A preferred temperature range is between 42° C. and 45° C. inclusive, and it is preferred that the formulation remain above 32° C. for at least 10 minutes, and preferably for 15 minutes or more, and preferably up to 30 minutes and more, including up to and in excess of 60 minutes.

The penetration enhancer may be in either formulation, provided that it at least a portion thereof is able, together with the drug, to contact the skin of the subject during thermogenesis. There may be more than one penetration enhancer, such as arlasolve and methanol, and each may be present in one or both formulations where there is a second formulation. Where there is a second enhancer, while it is preferred that it, and any further enhancers, is an enhancer as described, it may also be any penetration enhancer known in the art. Ethanol, Transcutol P, arlasolve, and methanol are separately preferred penetration enhancers, with combinations of any two or more thereof also being preferred.

In a preferred embodiment, there is provided a formulation comprising a supercooled solution of a salt, for use in the transdermal administration of a drug, said salt being capable of releasing heat of crystallisation and wherein said drug is administered as part of said formulation or as a second formulation, a penetration enhancer being present in one or both formulations, wherein said penetration enhancer is a C₁₋₁₂ compound comprising at least one -Alk-O— group, in which Alk is a C₁₋₆ alkylene and, when there is only one -Alk-O— group, then All may also represent a C₁₋₁₀ alkyl group.

In a further preferred embodiment, the thermogenic formulation utilises heat of hydration, and is achieved by mixing water, or other suitable solvent, with the salt, which term includes metal salts, oxides and/or hydroxides, to be hydrated, either in situ, or immediately before use and applying the resulting formulation to the patient. Suitable salts are generally anhydrous salts, and preferred such salts include the anhydrous forms of the salts calcium chloride (CaCl₂) and magnesium sulphate (MgSO₄). Other suitable salts include metal salts, oxides and/or hydroxides, and suitable such salts are disclosed in U.S. Pat. No. 4,338,098.Specific examples include calcium oxide, magnesium oxide

In a further preferred embodiment, the thermogenic formulation relies on heat of polymerisation, or enthalpy of polymerisation, such as by combining bisphenol-A (BPA) and epichlorohydrin (ECH).

The penetration enhancer may be a C₁₋₁₀ alkanol or, preferably, a C₁₋₆ alkanol, for example. The -Alk-O— may, in another embodiment, be an H-Alk-O— group, such as H-Alk-OH, or -Alk-O-Alk- and corresponding oligomers, optionally with one or more monomeric units, such as methoxy and ethoxy ether units. One example is Transcutol (diethylene glycol monoethyl ether). The -Alk-O— containing permeation enhancer may also have a saturated or partially unsaturated ring structure substituted by one or more -Alk-O— moieties, such as menthol and arlasolve (dimethyl isosorbide).

In general, it is preferred that the penetration enhancer is a C₁₋₆ alkanol.

In an alternative aspect, the present invention provides a formulation comprising a penetration enhancer as defined above, and a thermogenic formulation, such as a supercooled solution of a salt, for use in the transdermal administration of a drug, said salt being capable of releasing heat of crystallisation.

As noted above, the essential ingredients may all be present in a single formulation, or may be present in two/or more separate formulations that are mixed at, or prior to, application. In general, it is preferred to keep the salt solution as pure as possible in order to minimise any possibility of unwanted crystallisation prior to treatment.

Formulations of the invention are also referred to as salt solutions, salt formulations, and the like, herein, but it will be appreciated that such reference encompasses all formulations of the invention unless otherwise apparent from the context.

It will be appreciated that formulations that do not rely on supercooled salt will often only be generated in situ, or only very shortly before use, as the simple act of creating a formulation or solution may often be sufficient to trigger thermogenesis.

It is a particular advantage that the thermogenic formulations of the present invention have a heat release profile that, in combination with a lower alcohol, for example, can substantially enhance transdermal delivery of a drug, locally or systemically, over short time frames, from within 1 minute to continuous uptake over 30 minutes, and more. This contrasts with the art, where levels of heat at around 40° C. only show significant transdermal delivery after several hours, which can lead to significant compliance issues, and is demonstrated in the accompanying Comparative Example, and in FIGS. 11-14.

A thermogenic formulation, such as one containing a salt solution, may be applied directly onto a drug that has been applied to the skin. In this embodiment, the drug will generally be formulated prior to application, and it is not critical to the invention what formulation is used to administer the drug, provided that the drug formulation is capable of dispensing drug directly onto the skin. Where the salt formulation also comprises the penetration enhancer, then it is necessary for the two formulations to be able to interface, thereby to permit the alkanol, for example, to permeate, at least to some extent, into the drug formulation.

Thus, for example, a drug formulation that is a cream may be applied to the skin, and a formulation of the invention layered thereover in one or more layers. The penetration enhancer in the formulation of the invention then permeates into the cream and, it is believed, through to the skin, while heat generated when crystallisation is initiated serves to warm the area of application.

Where the drug formulation, rather than the heat generating formulation, comprises the penetration enhancer, then the drug formulation may comprise, or be provided with, a barrier, such as an occlusive barrier, that may, for example, prevent egress of the penetration enhancer in a direction away from the skin, provided that the formulation of the invention is able to provide sufficient heat through the barrier to be able to enhance drug permeation of the skin. Thus, it is preferred that any such barrier is not an insulating barrier, and it is generally preferred that the drug formulation is heatable throughout its thickness by heat released by the salt-containing formulation when activated. For example, the supercooled salt may be formulated in a patch with the drug formulation separated by a physical barrier, such as an impermeable membrane. In this embodiment, it is preferred that both the salt solution and the drug formulation both contact the barrier. A seed formulation, suitable to nucleate the salt-containing formulation, may be provided adjacent to the salt-containing formulation and separated therefrom by a frangible membrane. The frangible membrane may be ruptured by application of suitable force such that the seed formulation is exposed to the salt-containing formulation. This nucleates the salt-containing solution to release heat. An example of this embodiment is shown in accompanying FIG. 15. A compartment, or reservoir, containing the salt-solution is shown as ‘supercooled solution’. A compartment containing the seeding agent is shown as ‘seed/nucleation point’, with the line between the two compartments representing a frangible membrane that can be ruptured, for example, by pressure on one or other of the salt-containing compartment or the seed compartment. These compartments abut the adhesive compartment via an impermeable membrane suitable that heat can be exchanged between the salt-containing compartment and the adhesive, without permeation of salt into the adhesive, or drug or enhancer into the salt solution. The adhesive may be exposable by removal of a protective backing, thereby enabling adhesion to the skin. Subsequent rupture of the frangible membrane serves to activate the salt solution, thereby heating the adhesive/drug/enhancer mixture to enhance dermal penetration of the drug. This embodiment may be used for any of the thermogenic formulations of the invention, whereby an essential component is retained separately prior to use, and then mingled with the thermogenic formulation after rupture of a frangible member that had previously prevented the co-mingling of all ingredients.

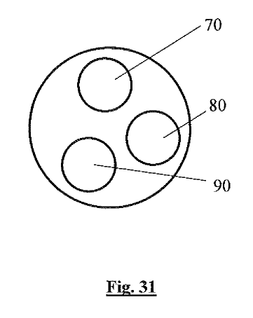

Further delivery embodiments are shown in FIGS. 30 and 31.

FIG. 30 shows a breakable container (20) comprises three frangible compartments (40, 50, 60), or reservoirs, one (50) containing the heat generating system, one compartment (60) containing the seeding agent is shown as ‘seed/nucleation point’, and the third compartment (40) containing the API and penetration enhancers all within an outer container (10). The compartments are formed from glass or other breakable material, where pressure on each container ruptures the container to release the contents. The formulation can be applied to the area of interest by positioning the applicator (30) over the target site.

A patch comprising three compartments is shown in FIG. 31. A compartment, or reservoir, containing the salt solution is shown at (80). A compartment containing the seeding agent is shown at (70). A compartment containing the API and penetration enhancer is shown at (90). All contained within a larger patch which has an adhesive and a permeable membrane on one side to allow for transfer of the API and penetration enhancer to the target site. The three compartments are made from a frangible membrane that can be ruptured allowing the contents to be mixed.

It will be appreciated that the enhancer may be present in the salt solution or in the seed preparation. In a patch embodiment, it will be appreciated that the enhancer must mix with the drug on activation of the salt solution. This may be effected by providing frangible or permeable barriers between the solutions, thereby enabling access to the drug by the enhancer on activation and/or the enhancer may be present in the adhesive used to secure the patch to the skin.

Where an embodiment of the invention employs an adhesive, and that adhesive is suitable to serve as a vehicle for a penetration enhancer, then it will be appreciated that the adhesive can serve as a formulation of the present invention.

Suitable substances to seed the salt solution are described elsewhere herein, but may include salt or any other suitable nucleant, such as crystals or other solids suitable to catalyse crystallisation.

Where the heat generating layer comprises the penetration enhancer, then it is generally preferred that the drug formulation, after application, forms a thin layer, so as not to act as a barrier for penetration enhancer reaching the skin. It is generally preferred to use patches where, for example, the two formulations are one and the same and can be loaded into the patch, or where the formulation of the invention is provided in a separate reservoir within the patch, and may be separated by a semi-permeable or frangible membrane, for example, as exemplified above.

In another embodiment, the two or more formulations are kept separate and then mixed prior to application.

As used herein, ‘thermogenic formulation’, ‘formulation of the invention’ and ‘heat generating formulation’ refer to formulation comprising the salt, while the ‘drug formulation’ refers to a formulation comprising a drug.

The drug formulation, which may include the patch adhesive, will normally be separate from the formulation of the invention, as discussed below. Suitable drug formulations that are preferred for use with the formulations of the invention include ointments, creams, gels, lotions, pastes, sprays, foams, and liniments, and generally any formulation in a liquid, semi-solid or spreadable form that is preferably not associated with an impermeable barrier, especially when the heat generating formulation comprises the penetration enhancer.

Other suitable delivery forms include dual spray formulations, wherein one formulation is sprayed on to the skin prior to another, although concurrent administration is contemplated by the present invention, with the drug formulation preferably being administered first, in order to derive maximal benefit from the heat released by the salt solution, and dual sachet formulations, whereby the contents of two sachets can be mixed in situ.

In an alternative aspect, there is provided a medicament for the transdermal administration of a drug, said medicament comprising separately disposed formulations, a first thermogenic formulation, such as aformulation comprising a supercooled solution of a salt, and a second formulation comprising drug to be topically administered, one or both of said formulations comprising a penetration enhancer, and wherein said salt is capable of releasing heat of crystallisation. The enhancer is as defined above. Other thermogenic formulations are as described above.

In a further aspect, there is provided a medicament for the transdermal administration of a drug, said medicament comprising separately disposed formulations, a first formulation comprising drug to be topically administered and a second thermogenic formulation comprising a penetration enhancer and a thermogenic ingredient or ingredients, such as a supercooled solution of a salt, being capable of releasing heat, such as heat of crystallisation. The enhancer is as defined above. Other thermogenic formulations are as described above.

There is further provided a method for enhancing transdermal administration of a drug, comprising localised heating of an area of skin where it is desired to apply drug, heating said area to between 40° C. and 50° C. inclusive, preferably 42° C. to 45° C. inclusive, for a period of between 1 minute and 60 minutes, and applying a formulation to said area, either during heating or immediately subsequent thereto, said formulation comprising said drug and at least one permeation enhancer as described above.

A kit comprising a medicament of the invention preferably further comprises means for initiating thermogenesis in the thermogenic formulation, such as crystallisation.

The term ‘transdermal administration’ is used to indicate penetration of the skin, and includes both penetration and crossing of the Stratum corneum, as desired. Both local and systemic administration are contemplated by this term.

The term ‘drug’ is used to denote any physiologically active substance that it is desired penetrate or cross the skin.

Without being restricted by theory, it is believed that the formulations of the invention have their effect by the penetration enhancer disrupting the lipids in the Stratum corneum and by the heat enhancing this effect, as well as the penetration enhancer providing a drug vehicle. Whatever the mechanism, the Examples provided hereinunder illustrate that the combination of heat and penetration enhancer is as much as ten times more effective than either heat or penetration enhancer alone in enhancing drug permeation across the skin.

Where the penetration enhancer is a lower alkanol, suitable examples include; methanol, ethanol, propan-1propan-2-ol (isopropyl alcohol, IPA), butan-1-ol, butan-2-ol, 2-methylpropan-2-ol, pentan-1-ol, pentan-2-ol, pentan-3-ol, 2,2-dimethylpropan-1-ol, 3-methybutan-1-ol, hexan-1-ol, hexan-2-ol, hexan-3-ol, 3,3 -dimethylbutan-1-ol, 3 -ethylbutan-1-ol, 4-methylpentan-1-ol, as well as the corresponding diols and triols. The monohydric compounds are preferred, particularly the straight chain alkanols. More preferred are the C₁₋₃ alkanols, especially methanol, ethanol and IPA. Most preferred in this group is ethanol. Individually, each of menthol and Transcutol P [2-(2-ethoxyethoxy)ethanol] is also preferred.

The formulations of the invention may comprise any salt (or combination thereof) that is/are capable of forming a supercooled solution and releasing heat of recrystallisation when exiting the supercooled state. Salts that may be used are typically salt hydrates, and may be selected from: LiClO₃.3H₂O, NaOH.3.5H₂O, KF.4H₂O, CaCl₂.6H₂O, Na₂SO₄.10H₂O, Na₂CO₃.10H₂O, CaBr₂.6H₂O, Na₂HPO₄.12H₂O, Zn(NO₃)₂.6H₂O, KF.2H₂O, Ca(NO₃)₂.4H₂O, Na₂S₂O₃.5H₂O, Na₂HPO₄.7H₂O, CH₃COONa.3H₂O, Cd(NO₃)₂.4H₂O, NaOH.H₂O, Na₂B₄O₇.10H₂O, Na₃PO₄.12H₂O, Al(NO₃)₂.9H₂O, Ba(OH)₂.8H₂O, KAl(SO₄)₂.12H₂O, Al₂(SO₄)₃18H₂O, Mg(NO₃)₂.6H₂O, NH₄Al(SO₄)₂.12H₂O, and MgCl₂.6H₂O. The most preferred salts for use in the present invention are sodium thiosulphate and/or sodium acetate, preferably sodium acetate.

A supercooled solution of salt may be prepared by any suitable means. In general, a supercooled solution may be prepared by heating the salt beyond its melting point in the presence of water, or other appropriate solvent, water being preferred. The solvent will be referred to herein as water, but it will be appreciated that this term also includes reference to other suitable solvents, unless otherwise apparent from the context. The water may be added after heating, in which case the water should be at least at the melting point of the salt, or the salt may be mixed with the water and heated together therewith until all salt is in solution. The solution may then be mixed with any further components, such as an alkanol, and cooled. Further components may be mixed after cooling, but this is not preferred, as there is a risk that crystallisation will occur.

Crystallisation in a supercooled salt solution may be induced by any suitable means, such as breaking of a frangible seal between two liquids, flexation of a disc or other material if the supercooled solution is in a patch, or simply by sprinkling on a preparation of the powdered salt (either powder of the anhydrous or hydrous forms), or other substance suitable to nucleate the supercooled salt, or by inducing crystallisation in the preparation prior to applying, or upon actuation, and optionally by manual mixing, although the administration form must take account of the potentially rapid crystallisation under such circumstances.

Crystallisation may also be induced by incorporating a suitable nucleant in the drug formulation. This may either be provided as a formulation suitable for use with the formulations of the invention, or the nucleant may be admixed with the drug formulation prior to administration to the skin. Thus, when the formulation of the invention is applied to the drug formulation, crystallisation and heating is homogeneous where applied.

Other excipients may also be incorporated in the formulations of the invention, and may include thickeners, pH modifiers, anti-oxidants, antimicrobials, co-solvents, and non-solvents, as well as the drug itself, if it is compatible with the supercooled salt solution. Other excipients may be used, as desired. In this sense, the term ‘compatible’ is used to mean that the presence of the drug will not lead immediately lead to significant crystallisation of the salt.

Thickeners include: such compounds as polyethylene glycol; gels, such as CMC; gums, such as xanthum gum; and starch. These may be used to control the rate of permeation in some formulations, for example, or to stop the formulation escaping once applied.

Antimicrobials include benzyl alcohol, for example, but many suitable compounds and substances are known in the art.

Suitable pH modifiers include any substances capable of modifying pH without causing crystallisation, and may be used to ensure that the pH of the formulation of the invention is such as to prevent any selected substance present in the formulation from crystallising out.

While the present invention contemplates the presence of drug in the heat generating formulation, this is generally not preferred, as many drugs are not readily soluble in such formulations, and also because many drugs are thermolabile, and it may be necessary to reheat the formulation of the invention in the event that crystallisation occurs prior to use. In one embodiment, the salt is not provided as a supercooled solution, but is provided as a solid together with water and any other ingredients as a pre-mix, which is heated prior to use to force the salt into solution. Once cool enough, the resulting formulation may be used as described herein.

The formulations of the invention are exemplified herein, and may include patches, as well as liquids, gels or other semi-solid formulations, and may be dispensed by squeezable tube, liquid dispenser or aerosol, for example.

Where the thermogenic formulation is a supercooled salt, the concentration of the salt solutions of the invention may be any that are desired, but will typically be chosen such as to deliver a desired amount of heat for a desired duration. This may be effected by the choice of concentration, as well as by other excipients that may be selected such as to reduce the speed of crystallisation, which may lengthen the overall crystallisation time and reduce the peak temperature reached.

The supercooled salt solution will typically form the majority of the sum of the formulations of the invention, and typically forms between 50 and 95% w/v of the formulation, more preferably between 70 and 90%, preferably about 75 to about 85%.

The penetration enhancer may be present in an amount of about 1 to about 40% w/v, preferably about 1 to about 30% w/v, more preferably about 5 to about 20%. An amount of about 15% has been found to be useful when the penetration enhancer is a C₁₋₆ alkanol, for example.

Drugs that may be administered transdermally in accordance with the present invention include, but are not limited to, any one or more of antivirals, such as 1-docosanol, abacavir, aciclovir, acyclovir, adefovir, amantadine, amprenavir, arbidol, atazanavir, atripla, boceprevir, brivudine, cidofovir, combivir, darunavir, delavirdine, didanosine, docosanol, edoxudine, edoxudine, efavirenz, emtricitabine, enfuvirtide, entecavir, entry inhibitors, famciclovir, fixed dose combination (antiretroviral), fomivirsen, fosamprenavir, foscarnet, fosfonet, fusion inhibitor, ganciclovir, ibacitabine, idoxuridine, imiquimod, imunovir, indinavir, inosine, integrase inhibitor, interferon, interferon type i, interferon type ii, interferon type iii, lamivudine, lopinavir, loviride, maraviroc, moroxydine, nelfinavir, nevirapine, nexavir, nucleoside analogues, oseltamivir (tamiflu), peginterferon alfa-2a, penciclovir, peramivir, pleconaril, podophyllotoxin, protease inhibitor (pharmacology), raltegravir, resiquimod, reverse transcriptase inhibitor, ribavirin, rimantadine, ritonavir, saquinavir, stavudine, synergistic enhancer (antiretroviral), tenofovir, tenofovir disoproxil, tipranavir, trifluridine, trizivir, tromantadine, truvada, valaciclovir (valtrex), valganciclovir, vicriviroc, vidarabine, viramidine, zalcitabine, zanamivir (relenza), zidovudine, and pharmaceutically acceptable salts of such drugs; drugs suitable to treat acne, including topical antibacterials, retinoids and related preparations, such as acitretin, adapalene, azelaic acid, bexarotene, benzoyl peroxide, calendula, chlorhexidine gluconate, clindamycin, dithiosalicylic acid, erythromycin, isotretionoin, motretinide, polprenoic acid, resorcinol, tazarotene, tetracycline, tretinoin, triclosan; drugs suitable to treat autoimmune diseases, including i) those suitable for the treatment of Alzheimer's, such as donepezil hydrochloride, galantamine, memantine hydrochloride, rivastigmine, and pharmaceutically acceptable salts thereof, ii) glucocorticoids, such as hydrocortisone (cortisol), cortisone acetate, prednisone, prednisolone, methylprednisolone, dexamethasone, betamethasone triamcinolone, beclometasone, fludrocortisone acetate, deoxycorticosterone acetate (doca), and aldosterone, iii) cytostatics (classic alkylating), such as nitrogen mustards, cyclophosphamide, mechlorethamine or mustine (hn2), uramustine or uracil mustard, melphalan, chlorambucil, ifosfamide, nitrosoureas, carmustine, lomustine, streptozocin, alkyl sulfonates, and busulphan, iv) cytostatics (alkylating like), such as platinum, cisplatin, carboplatin, nedaplatin, oxaliplatin, satraplatin, and triplatin tetranitrate, v) cytostatics (non-classic), such as procarbazine, altretamine, tetrazines, dacarbazine, mitozolomide, dacarbazine, and temozolomide, vi) cytostatics such as methotrexate, methotrexate sodium, methotrexate disodium, azathioprine, anthracyclines, mitomycin c, bleomycin, and mithramycin, vii) anti-metabolites, such as purine analogues, pyrimidine analogues, and antifolates, and viii) immunophilins, such as ciclosporin, tacrolimus, sirolimus; antihistamines, including i) general anthistamines such as antazoline, chlorcyclizine hydrochloride, dimethindene maleate, diphenhydramine, histapyrrodine, isothipendyl hydrochloride, mepyramine, mepyramine maleate, tolpropamine hydrochloride, tripelennamine hydrochloride, and triprolidine hydrochloride, ii) h1-receptor antagonists, such as diphenhydramine, loratadine, desloratadine, meclizine, fexofenadine, pheniramine, cetirizine, promethazine, chlorpheniramine, and levocetirizine, iii) h2-receptor antagonists, such as cimetidine, famotidine, ranitidine, nizatidine, roxatidine, and lafutidine, iv) h3-receptor antagonists, such as a-349,821, abt-239, ciproxifan, clobenpropit, and thioperamide, and v) h4-receptor antagonists, such as thioperamide, jnj 7777120, and vuf-6002; corticosteroids, such as alclometasone dipropionate, beclomethasone dipropionate, betamethasone valerate, clobetasol propionate, clobetasone butyrate, desoximetasone, diflucortolone valerate, fludroxycortide/flurandrenolone, fluocinolone acetonide, hydrocortisone, hydrocortisone acetate, and hydrocortisone butyrate; dermatological drugs, such as azathioprine, becaplermin, bentoquatum, ciclosporin, gamolenic acid, glycolic acid, ichthammol, hydrodquinone/mequinol, lithium succinate, keluamid, monobenzone, mycophenolate mofetil, polyphloroglucinol phosphate, sodium pidolate, sulphur, sulphurated lime, sulphurated potash, and minoxidil; parasiticidal preparations, such as benzyl peroxide, carbaryl, malathion, permethrin, and phenothrin; topical circulatory preparations, such as heparinoid; transdermal drugs, such as ibuprofen, diclofenac, glyceryl trinitrate, oxybutynin, nicotine, ethinylestradiol+norelgestronin, griseofulvin, hyoscine, alfentanil, fentanyl, remifentanil, testosterone, oestrogen, methylphenidate hydrochloride, and methyl predisolone; topical antifungals, such as 2-(4-chlorphenoxy)-ethanol, aluminium chloride, amorolfine, amphotericin b, benzoic acid, benzoyl peroxide, bifonazole, bromochlorsalicylanilide methylrosaniline, butenafine, butoconazole, buclosamide, butenafine hydrochloride, chlorophensesin, chlormidazole hydrochloride, chlorophenesin, ciclopirox, ciloquinol (iodochlorhydroxyquin), clotrimazole, croconazole, dimazole, eberconazole, econazole, ethyl hydroxybenzoate, fenticlor, fenticonazole, fluconazole, flucytosine, flutimazole, griseofulvin, haloprogin, isoconazole, itraconazole, ketoconazole, lanoconazole, mepartrician, miconazole, natamaycin, natrifine, neticonazole, nystatin, omoconazole, oxiconazole, pyrrolnitrin, polynoxylin, potassium iodide, potassium permanganate, salicylic acid, selenium sulphide, setraconazole, sulbentine, sulconazole, sodium propioniate, sulphacetamide, terbinafine, terconazole, ticlatone, tioconazole, tolciclate, tolnaftate, tribromometacresol, triacetin, undecylenic acid, undecenoates, zinc pyruthione, and pharmaceutically acceptable salts thereof; topical antibacterials, such as achromycin, amphomycin, azithromycin, bacitracin/bacitracin zinc, bekanamycin sulphate, benzo[f][1,7]naphtyridone derivatives, chloramphenicol, chloraquinaldol, chlortetracycline, dapsone, framycetin sulphate, fusidic acid, halquinol, mupirocin, neomycin sulphate, polymyxins, silver sulphadiazine, sulphanilamide, sulphasomidine, sulphathiazole, sulfonamides, tetracycline; NSAIDs, such as naproxen, ibuprofen, indomethacin, diclofenac, ketoprofen, piroxicam, etofenamat, fenbufen,fenoprofen, fluorbiprofen, mefenamic acid, meloxicam, celecoxib, etoricoxib, etodolac, celecoxib, acaproazone, tenoxicam,tiaprofenic acid,and pharmaceutically acceptable salts thereof; local anaesthetics, such as amethocaine, amylocaine, benzocaine, bucricaine, butacaine, butyl aminobenzoate picrate, cincocaine, dimethisoquin hydrochloride, dylocaine hydrochloride, ethyl chloride, lidocaine, lignocaine, myrtecaine, oxethazaine, prilocaine, propanocaine hydrochloride, tetracaine; drugs suitable to treat psoriasis, such as calcipotriol, coal tar, ciclosporin, dithranol, 5-fluouracil, fumaric acid, lonapalene, halquinol, mupirocin, neomycin sulphate, polymyxins (polymyxin b sulphate), silver sulphadiazine (sulphadiazine), sulphanilamide, sulphasomidine, sulphathiazole, desoximetasone, fluocinonide, and argan oil. other suitable drugs include the non-steroidal anti-inflammatories (NSAIDs), such as diclofenac, actinic keratosis treatments, and capsaicin. It will be appreciated that salts and any other recognised pharmaceutical forms of any of the above are also contemplated by the present invention.

Preferred transdermal drugs that have been approved for use include; scopolamine, nitroglycerin, clonidine, estradiol, fentanyl, nicotine, testosterone, lidocaine with epinephrine, estradiol with norethidrone, lidocaine, ethinyl estradiol with norelgestromin, estradiol with levonorgestrel, oxybutynin, lidocaine, lidocaine with tetracaine, fentanyl HCl, methylphenidate, selegiline, rotigotine, and rivastigmine. Other suitable drugs include, camphor, capsaicin, menthol, methyl salicylate, ethyl salicylate, diethylamine salicylate, glycol salicylate, triethyl amine salicylate, methyl nicotinate, triethanolamine nicotinate, ethyl nicotinate, hexyl nicotinate, phenyl nicotinate, benzyl nicotinate, alpha tocopherol nicotinate, and levamenthol.

It will be appreciated that the present invention further provides a physiologically acceptable formulation comprising a penetration enhancer and a thermogenic system, such as a supercooled solution of a salt, said system being capable of releasing heat, such as heat of crystallisation. Such formulations are preferably as described hereinabove.

The term ‘physiologically acceptable’ refers to formulations that are acceptable for a physician to recommend or dispense for dermal application. In the event that there are contraindications, then the term is indicative that the benefits should outweigh the disadvantages. It is preferred that all formulations for use in the present invention are physiologically acceptable, where there is the intention or likelihood of the formulation coming into contact with the skin of a patient.

The present invention further provides a method for the transdermal administration of a drug, comprising topical administration of a first drug formulation, and subsequent administration of a second formulation comprising a thermogenic system, such as a supercooled solution of a salt, directly onto said first formulation, at least one of said formulations comprising a penetration enhancer, and wherein said system, such as the salt, is capable of releasing heat, such as heat of crystallisation, and where a salt has been caused to crystallise, causing said salt to crystallise, in situ.

In the accompanying Examples, there is shown that a 2.2 fold enhancement of lidocaine release was observed across a cellulose membrane when a supercooled sodium acetate solution containing several co-solvents was applied (maximum temperature achieved was 41° C.) compared to a control that did not generate heat. Repeating this experiment using silicone as the artificial skin barrier gave a 43 fold increase. Removing the non-polar solvents from the supercooled sodium acetate solution did not affect heat generation, but resulted in a maximum lidocaine permeation enhancement of 2.2 across the regenerated cellulose barrier and 11.7 for silicone. Repeating the experiment across human epidermal sheet resulted in a 76 fold increase in permeation after application of the formulation of the present invention in just ten minutes, compared to the control.

The data generated in the lidocaine mass transport experiments demonstrates that the combination of a supercooled salt solution with a drug enhances both the release from the topical formulation and permeation through a lipidic barrier. Furthermore, permeation can be further enhanced with the addition of certain solvents to the supercooled formulations.

The accompanying Figures are for illustration only, and are as follows:

FIG. 1 shows temperature profiles through the cellulose membrane after the application of an acetate solution above 1 ml 15 mg/ml lidocaine CMC (2.5% w/w) gel. Formulation of the invention (□), F-Heat (∘), F-PEs (×) and Control (Δ). Each point represents the mean±SD (n=3);

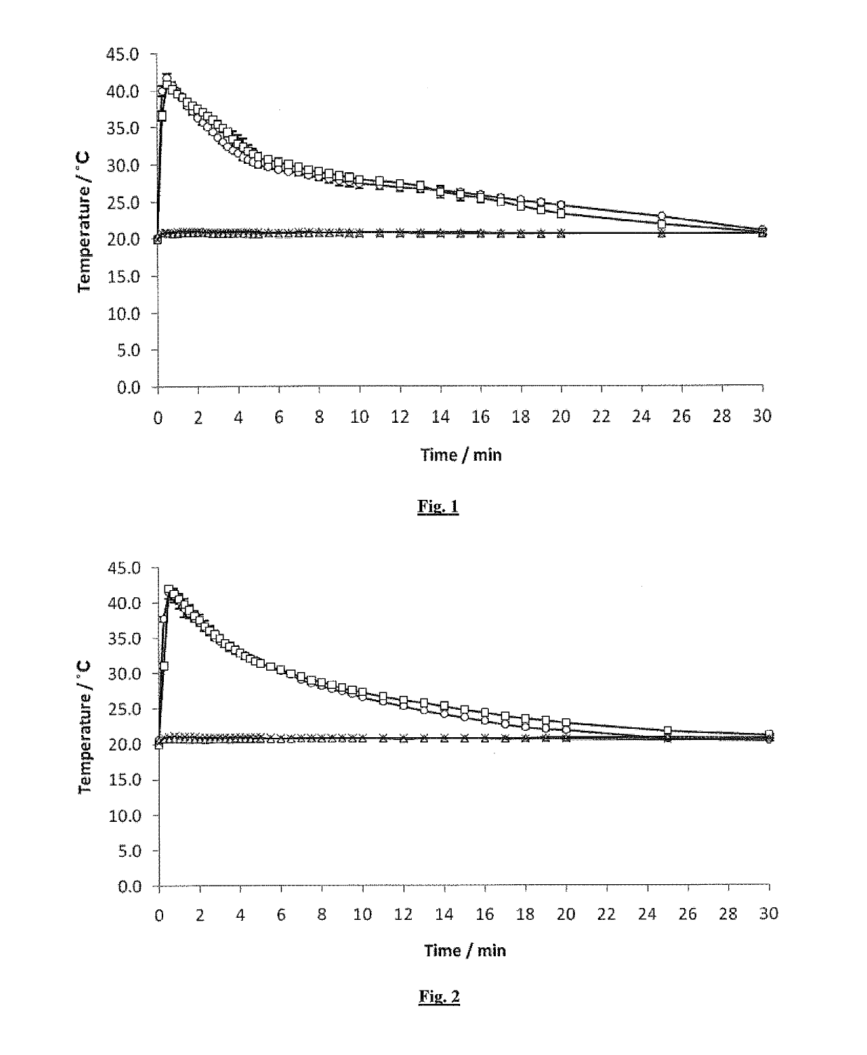

FIG. 2 shows temperature profiles through silicone membrane after the application of an acetate solution above 1 ml 15 mg/ml lidocaine CMC (2.5% w/w) gel. Formulation of the invention (□), F-Heat (∘), F-PEs (×) and Control (Δ). Each point represents the mean±SD (n=3);

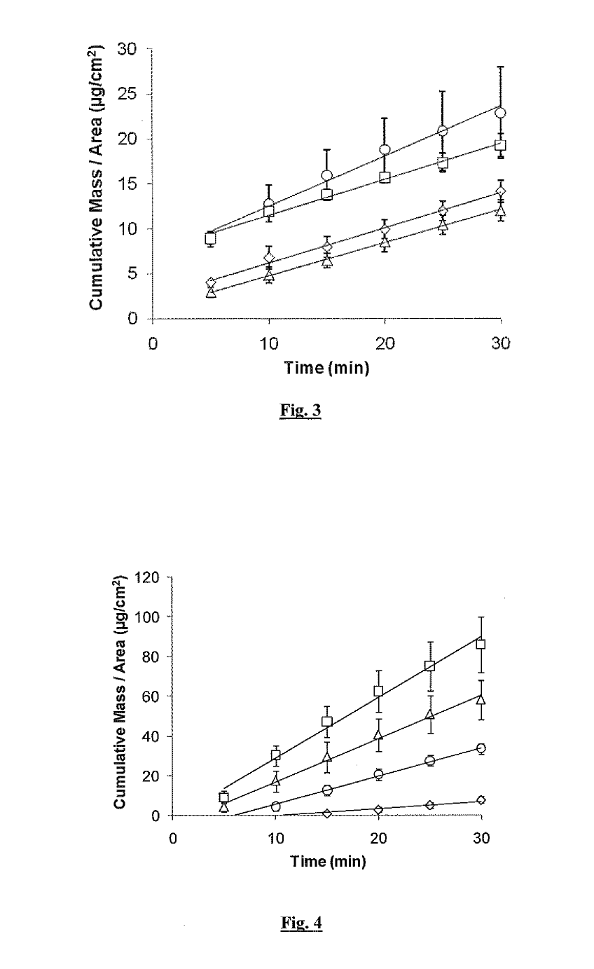

FIG. 3 shows lidocaine release through a regenerated cellulose membrane from 1.0 ml (15 mg/ml, pH 11.6, 2.5% CMC) gel with the formulation of the invention (□), F-Heat (∘), F-PEs (Δ) or the control (⋄). Diffusion cells were held in a thermoregulated vivarium to ensure an initial membrane temperature of 32° C. Each point represents the mean cumulative mass per area±standard deviation (n=5);

FIG. 4 shows lidocaine permeation through silicone membrane from 1.0 ml (15 mg/ml, pH 11.6, 2.5% CMC) gel with the formulation of the invention (□), F-Heat (∘), F-PEs (Δ) or the control (⋄). Diffusion cells were held in a thermoregulated vivarium to ensure an initial membrane temperature of 32° C. Each point represents the mean cumulative mass per area±standard deviation (n=5);

FIG. 5 shows lidocaine permeation through human epidermal sheet from 1.0 ml (15 mg/ml, 2.5% CMC) lidocaine gel donor (pH adjusted) with either the formulation of the invention (□) or F-PEs (Δ), F-Heat (∘), or the control (⋄) (pH adjusted). Diffusion cells were held in a thermoregulated vivarium at 32° C. Each point represents the mean cumulative mass per area±standard deviation (n=5);

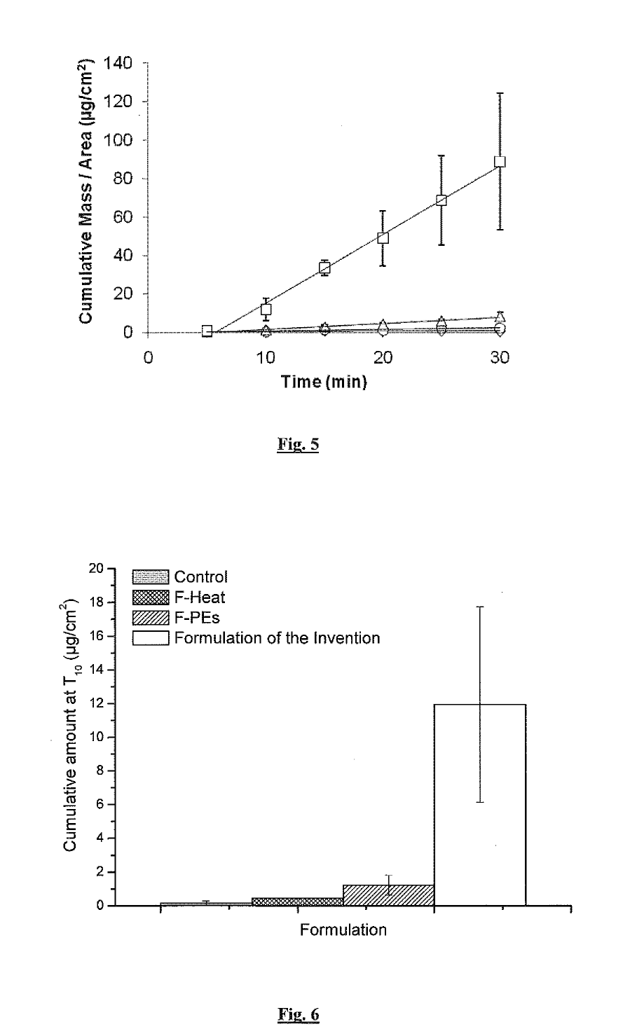

FIG. 6 shows the cumulative amount of lidocaine base (μg/cm²) released from a gel and acetate donor (pH adjusted) through human epidermal sheet after 10 minutes. The formulations refer to Control (Grey), F-Heat (cross-hatching), F-PEs (striped), Formulation of the invention (white). Each bar represents the mean standard deviation (n=5);

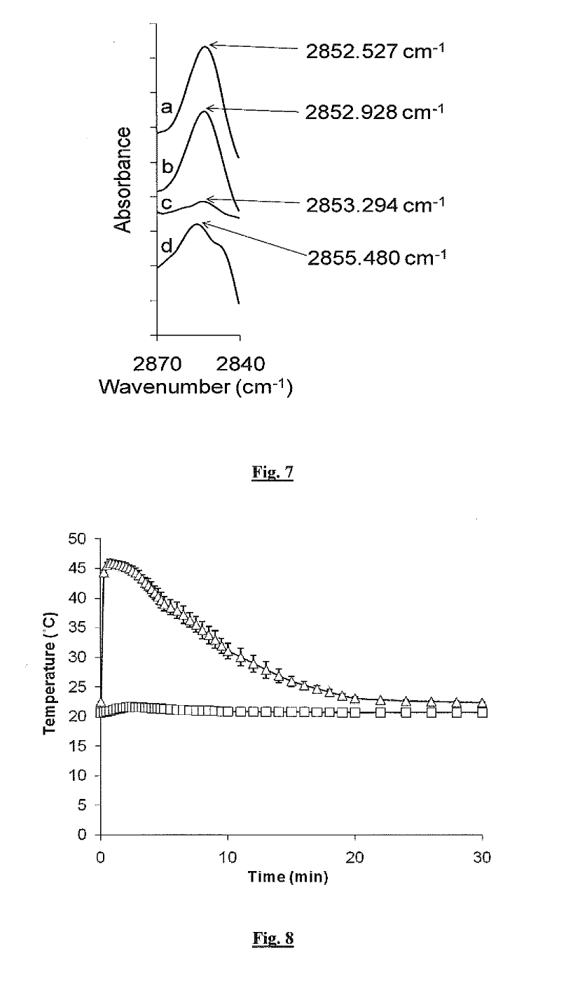

FIG. 7 shows ATR-FTIR spectra between 2870-2840 cm⁻¹ illustrating the shifts in the —CH₂ symmetric stretching region of human epidermal sheet after a 120 second application of Formulation of the present invention (d), F-PEs (c), F-Heat (b) and no formulation (Control, a) (n=1);

FIG. 8 shows temperature profiles measured over a 30 minute period for the formulation of the invention DS (Δ) and Control DS (□). Each point represents the mean temperature±standard deviation (n=3);

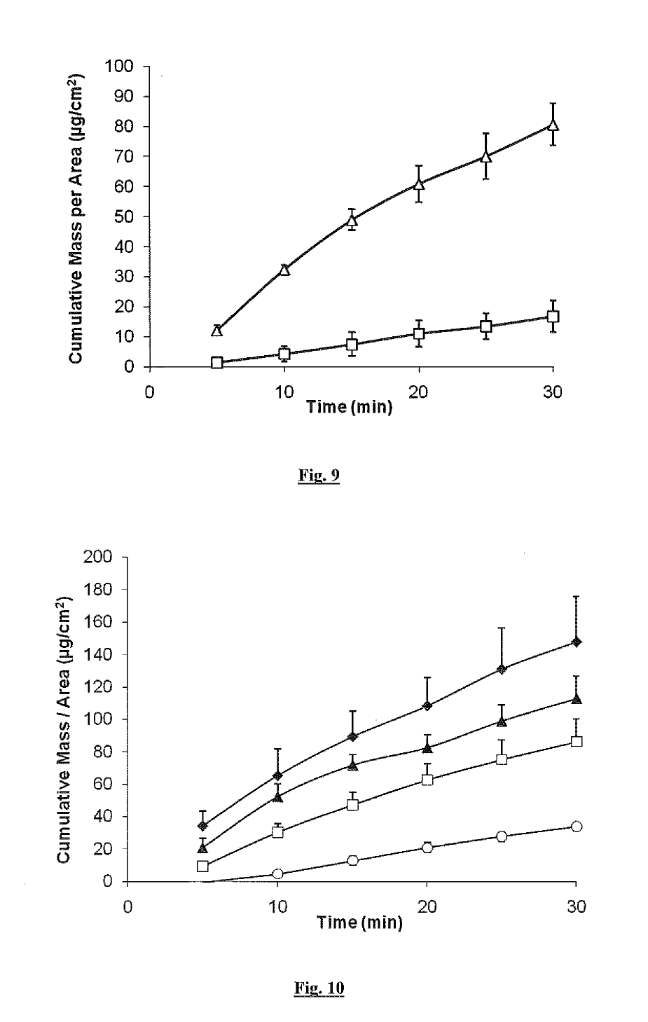

FIG. 9 shows cumulative permeation of lidocaine base from the formulation of the invention DS (Δ) and Control DS (□) through silicone membrane into phosphate buffered saline (0.15 M, pH 7.3). Cells were thermoregulated in a vivarium to ensure an initial membrane temperature of 32° C. Each point represents the mean cumulative mass per area±standard deviation (n=5);

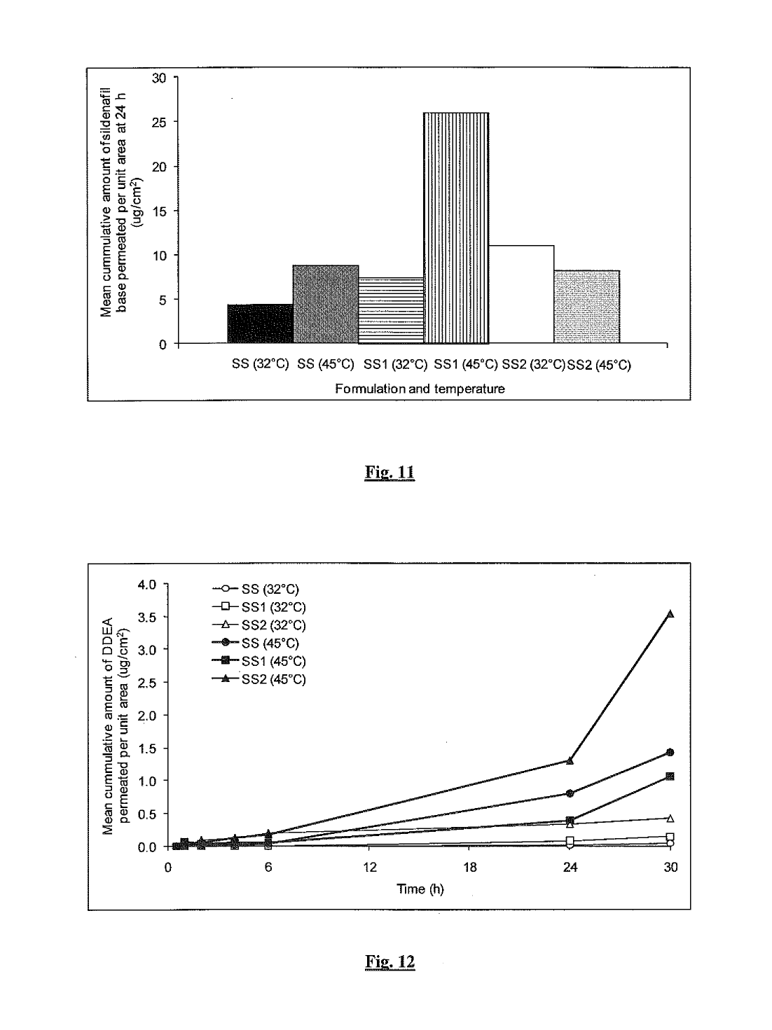

FIG. 10 shows lidocaine permeation through silicone membrane from 1.0 ml (15 mg/ml, pH 11.6, 2.5% CMC) gel with the formulation of the invention (□), F-Heat (∘), F-ΔAlcohol (▴) or the F-ΔEtOH (♦). Diffusion cells were held in a thermoregulated vivarium to ensure an initial membrane temperature of 32° C. Each point represents the mean cumulative mass per area+standard deviation (n=5);

FIG. 11 shows mean cumulative permeation of sildenafil base through human epidermal sheet at 24 hours from three test formulations at 32° C. and 45° C.;

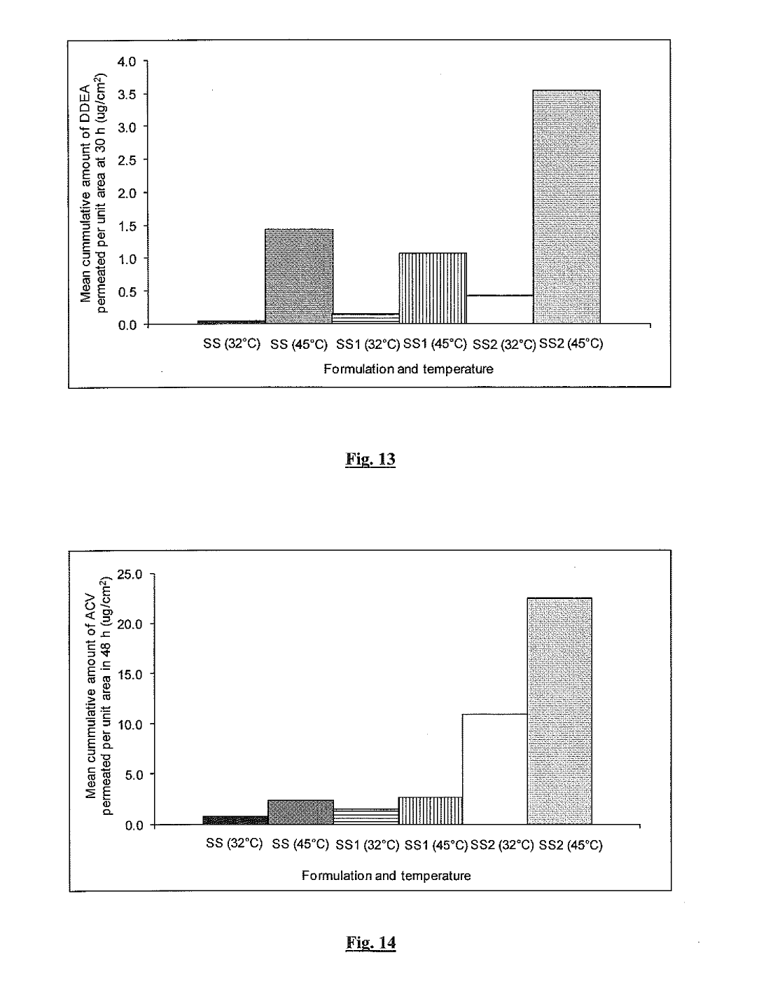

FIG. 12 shows mean permeation of DDEA through human epidermal sheet from three test formulations at 32° C. and 45° C. for the first 30 h of the permeation study;

FIG. 13 shows mean cumulative permeation of DDEA through human epidermal sheet at 30 hours from three test formulations at 32° C. and 45° C.;

FIG. 14 shows mean cumulative permeation of acyclovir through human epidermal sheet at 48 hours from three test formulations at 32° C. and 45° C.;

FIG. 15 illustrates an embodiment of the invention;

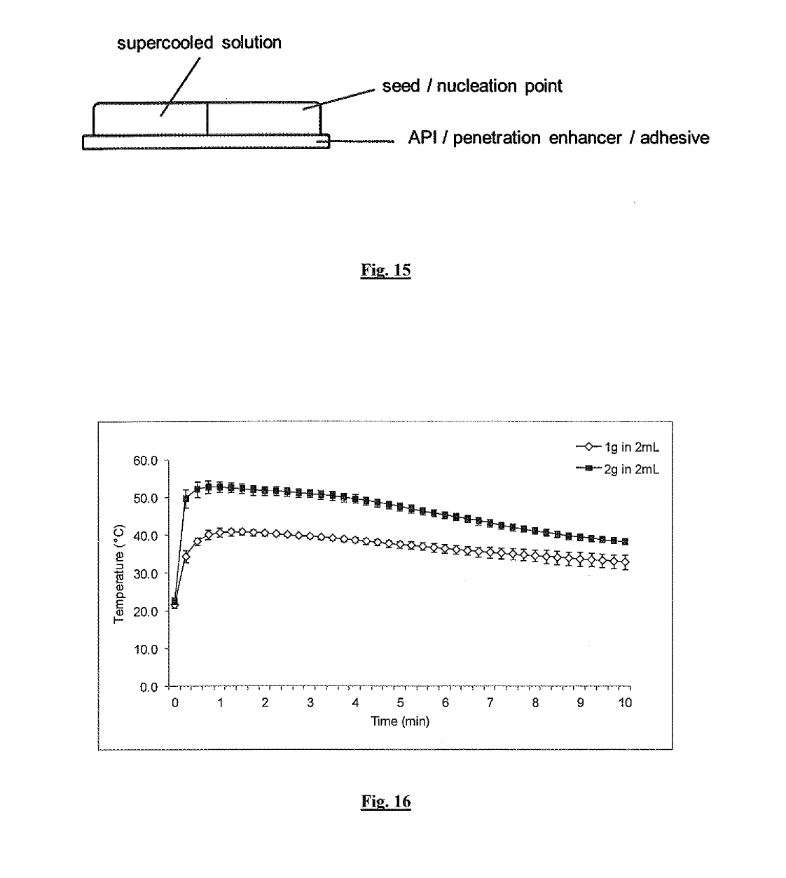

FIG. 16: Temperature profile from hydration of 1.0 g and 2.0 g of CaCl₂ in 2 mL of water (mean±SD, n=5);

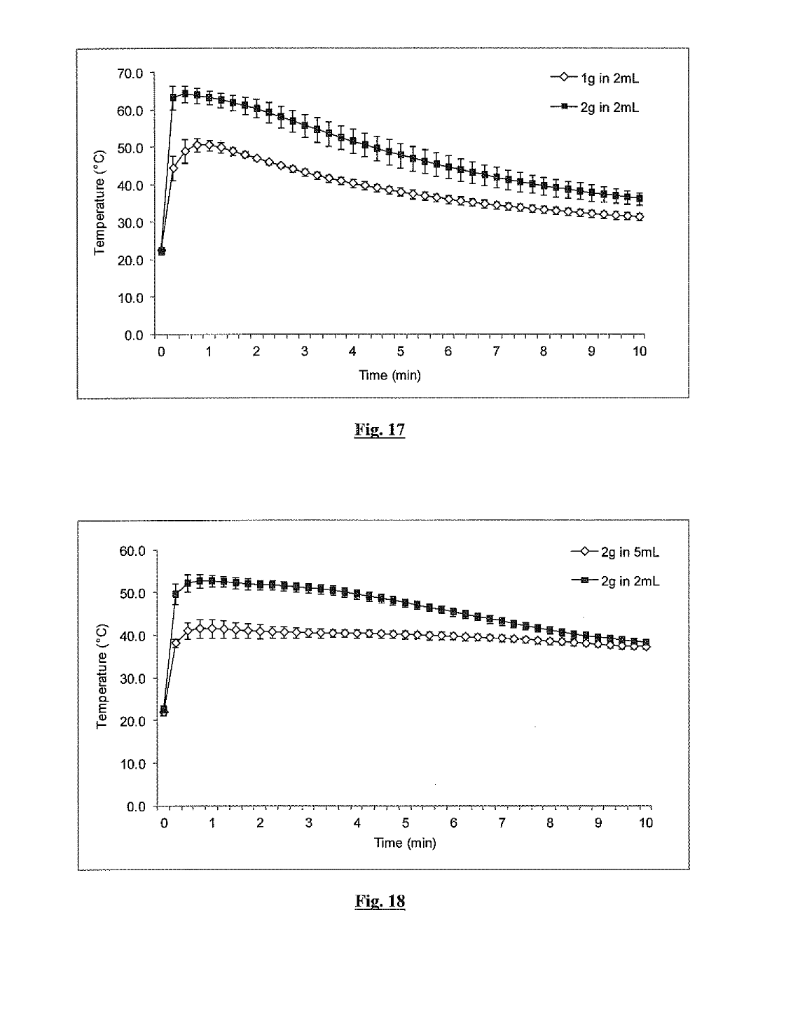

FIG. 17: Temperature profile from hydration of 1.0 g and 2.0 g of MgSO₄ in 2 mL of water (mean±SD, n=5);

FIG. 18: Temperature profile from hydration of 1.0 g of CaCl₂ respectively in 2 and 5 mL of water (mean±SD, n=5);

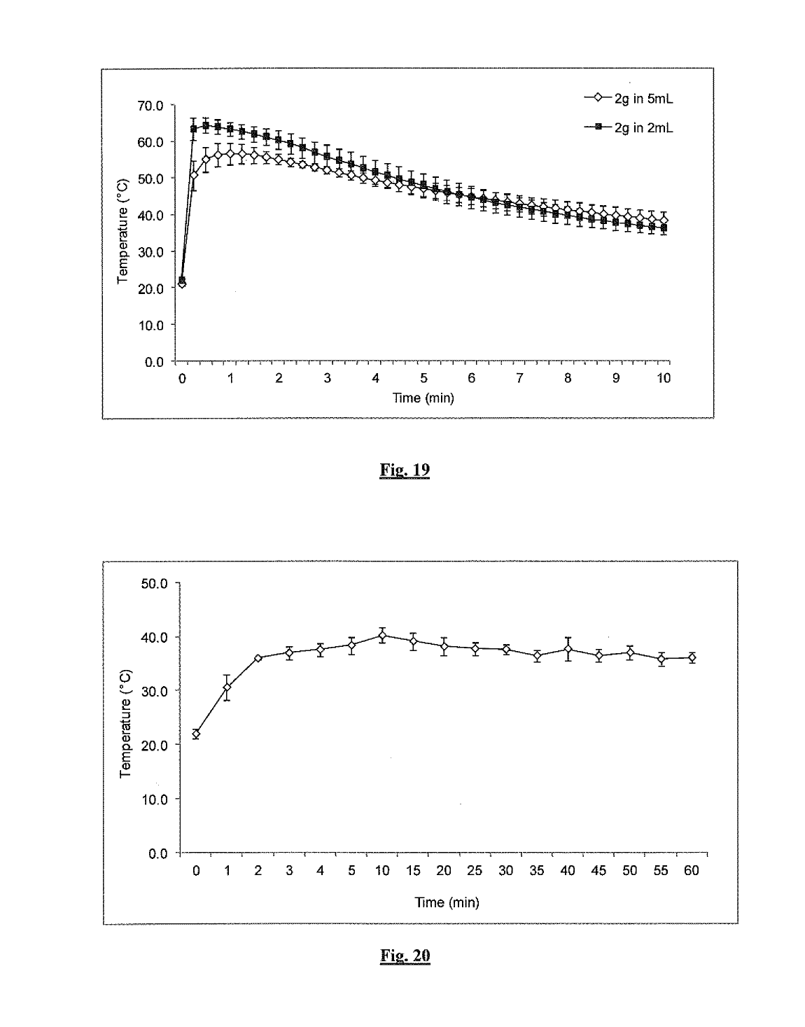

FIG. 19: Temperature profile from hydration of 1.0 g of MgSO₄ respectively in 2 and 5 mL of water (mean±SD, n=5);

FIG. 20: Temperature profile from polymerisation reaction, initiated in the presence of BPA and ECH (mean±SD, n=5);

FIG. 21: Mean permeation profile of sildenafil citrate through epidermal sheet from six donor formulations at 32° C. and 45° C. for 4 hours, n≧3;

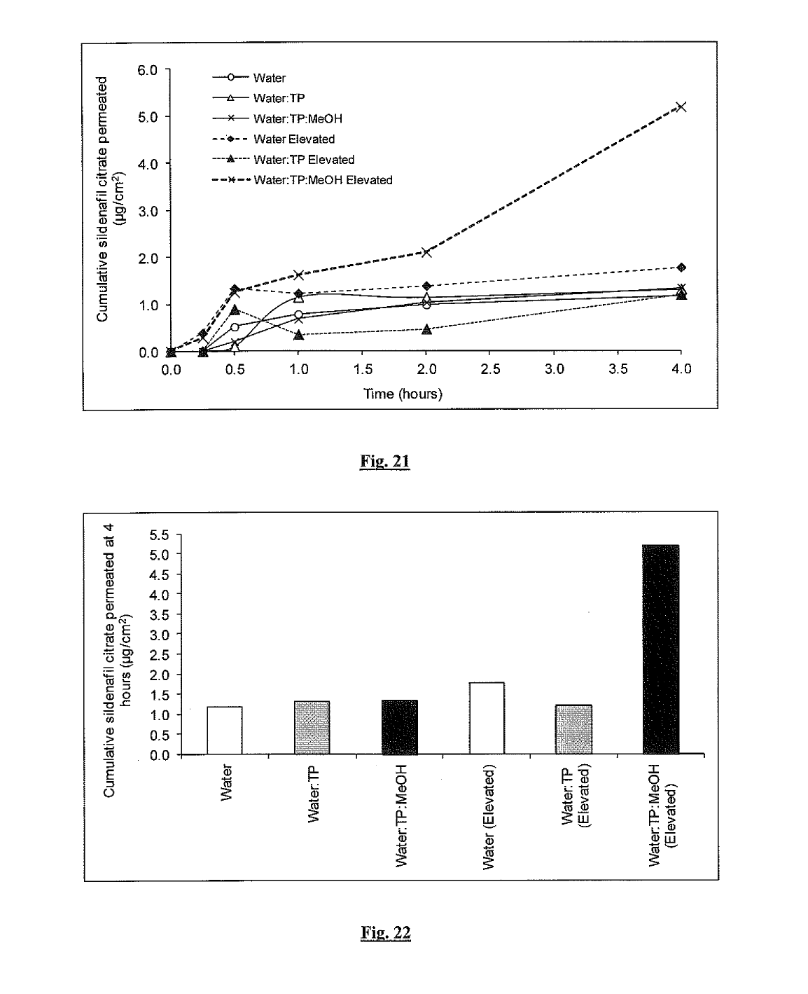

FIG. 22: Mean cumulative permeation of sildenafil citrate (μg/cm²) through human epidermal sheet at 4 hours from three donor formulations at 32° C. and 45° C., n≧3;

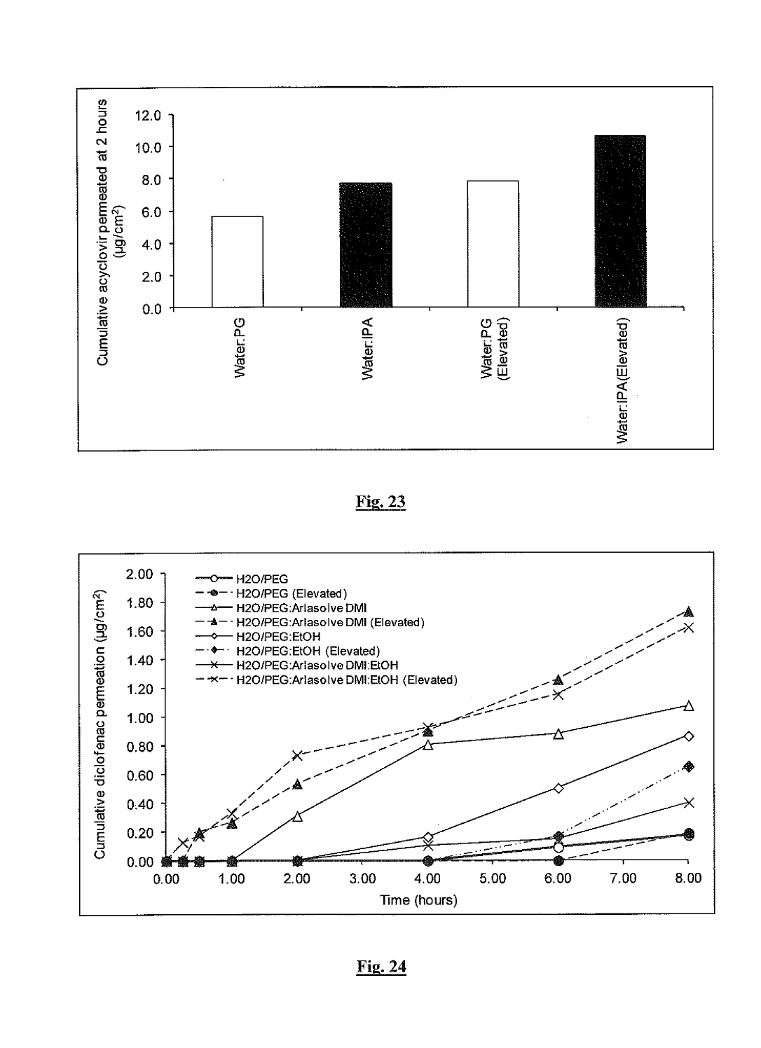

FIG. 23: Mean permeation profile of Acyclovir through epidermal sheet from four donor formulations at 32° C. and 45° C. for 2 hours, n≧3;

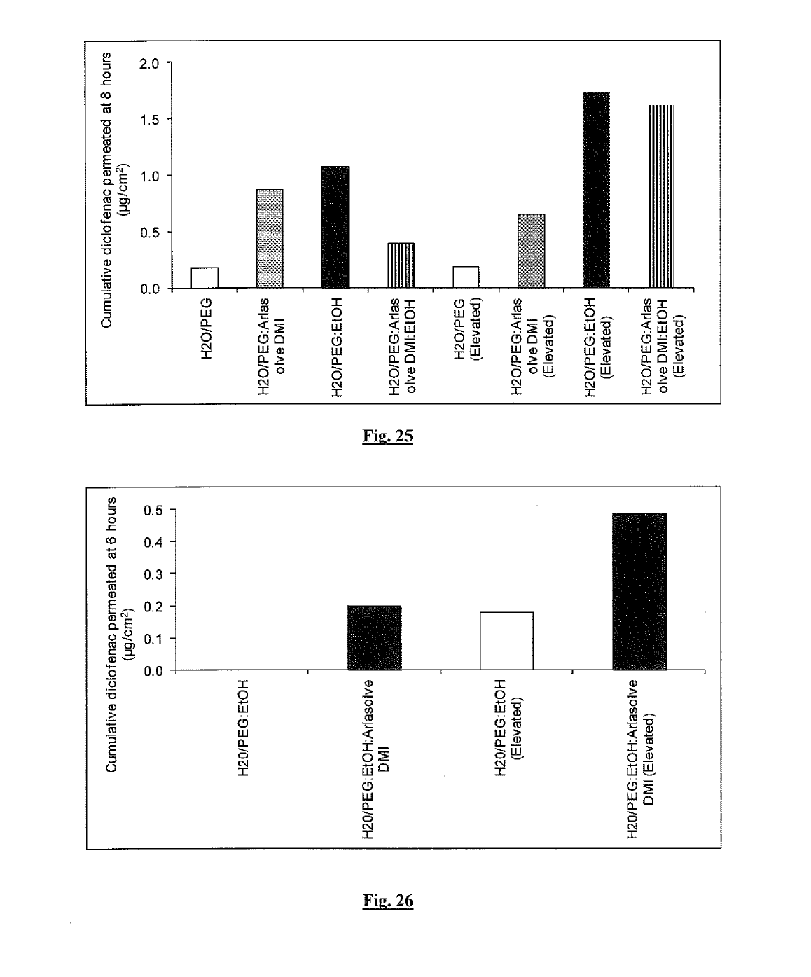

FIG. 24: Mean cumulative permeation profile of diclofenac formulations through human epidermal sheet from eight donor formulations at 32° C. and 45° C. for 15 minutes, n≧4;

FIG. 25: Mean cumulative permeation of diclofenac free acid (μg/cm²) through human epidermal sheet at 8 hours from four donor formulations at 32° C. and 45° C., n≧4;

FIG. 26: Mean cumulative permeation of diclofenac free acid (μg/cm²) through human epidermal sheet from four donor formulations with heat generated from supercooled sodium thiosulphate, n≦3;

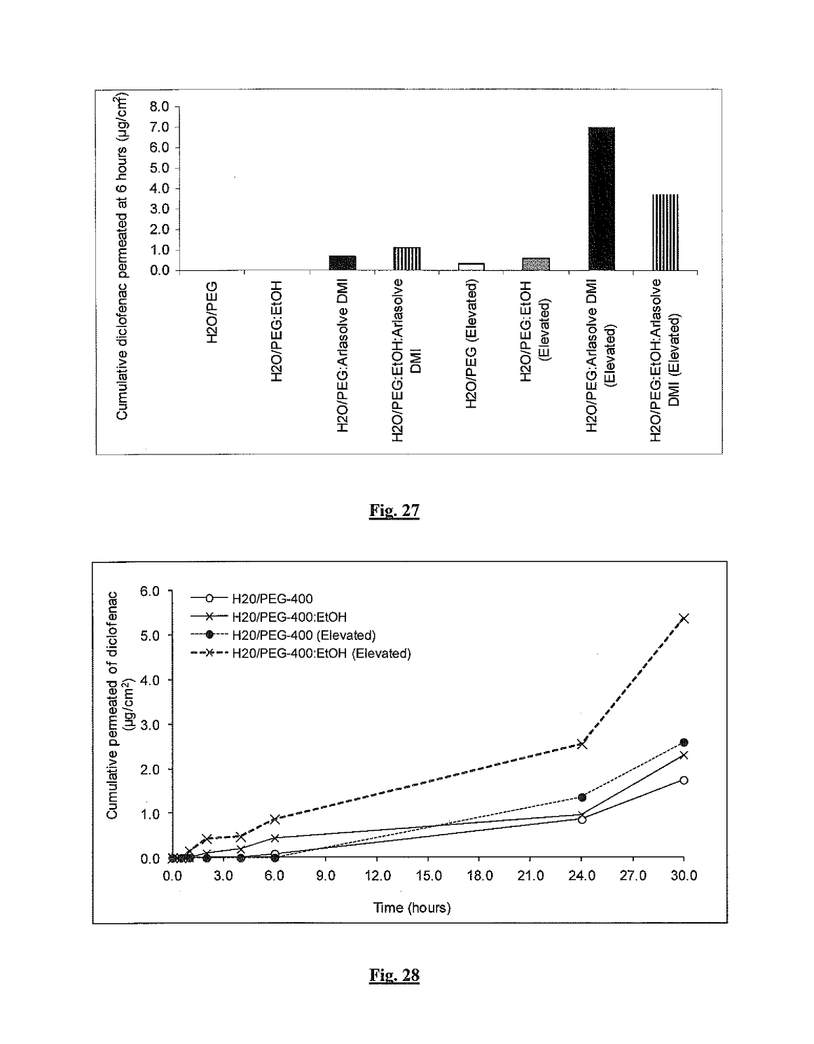

FIG. 27: Cumulative diclofenac permeation at 6 hours using the from eight donor formulations, with heat generated from supercooled sodium acetate, n≦3;

FIG. 28: Mean permeation profile of sildenafil citrate through epidermal sheet from four using the from 8 donor formulations, with heat generated from supercooled sodium acetate, n≧3;

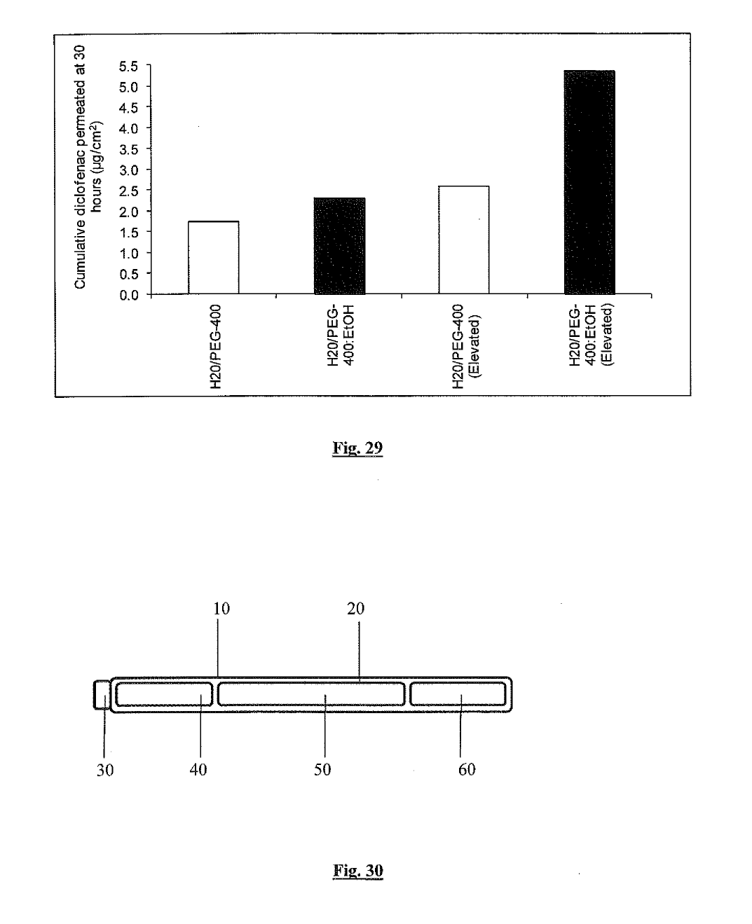

FIG. 29: Mean cumulative diclofenac permeation at 30 hours using the from four donor formulations, with heat generated from supercooled sodium acetate, n≧3;

FIG. 30: Container with three frangible internal containers; and

FIG. 31: Aerial view of a patch with three frangible compartments.

The following Examples illustrate the present invention and are not limiting thereon.

### EXAMPLES

### Materials and Methods

### Materials

Carboxymethylcellulose (CMC) sodium salt, medium viscosity (400-800 cps, 2% aqueous solution at 25° C.), benzyl alcohol, Pyrrolidine (99%), anhydrous potassium acetate and sodium acetate were all purchased from Sigma Aldrich (Gillingham, UK). PEG-400 (polyethylene glycol) was obtained from Merck (Darmstadt, Germany). HPLC grade methanol and deionised water were purchased from Fisher Scientific (Loughborough, UK). Lidocaine base (BP) was purchased from QueMaCo Ltd. chemical marketing company (Nottingham, UK). Regenerated cellulose membrane (RCM) in the form of dialysis tubing 12,000-14,000 MWCO size 5 was purchased from Medicell International (London, UK). Silicone membrane (Folioxane C16—Polydimethylsiloxane) 0.12 mm thickness was purchased from Kapitex Healthcare Ltd. (Yorkshire, UK). Phosphate buffered saline (PBS) (0.15 M, pH 7.3) was obtained from Oxoid (Basingstoke, UK). Ethanol (99%) was purchased from BDH (Leicestershire, UK). Super glue (ethyl cyanoacrylate) was supplied by Henkel Ltd. (Cheshire, UK).

### Methods

### Preparation of Membranes and Skin

### Regenerated Cellulose Membrane (RCM)

Regenerated cellulose received in the form of dialysis tubing was heated (70° C.) in deionised water for 1 hour. After heating the tubing was rinsed with cool deionised water to remove any impurities and was then cut to size.

### Silicone Membrane

No preparation was required for silicone membrane.

### Human Epidermal Sheet

Epidermal sheet (ES) from human abdominal skin was prepared in accordance with the method devised by Kligman and Christophers (1963). In brief, the skin was de-fatted and refrozen. Prior to preparation of epidermal sheets, the full thickness skin was defrosted at room temperature. Once thoroughly defrosted, the full thickness skin was placed into a glass beaker for 60 seconds containing deionised water at 60° C.±3° C. The skin was removed using forceps and placed dermal side down on aluminium foil. The epidermis was rolled off carefully using the thumb. The ES was floated on deionised water SC facing upwards whilst filter paper was floated underneath. The ES on filter paper was removed and frozen in aluminium foil until required. Prior to use in the permeation studies, the ES was defrosted for 1 hour then the filter paper backing was removed and the ES was mounted onto Franz cells.

### Example 1

### Preparation and Use of the Supercooled Solutions

### Supercooled Salt Formulations

Stock solutions were prepared by melting/dissolving either sodium or potassium acetate in water at 80° C. A 2.5 ml aliquot of the salt solution was transferred into a sample vial containing any other components of the preparations. The salt solutions were allowed to cool to room temperature. A total of three different test preparations and a control were constructed using the salt solutions (Table 1).

TABLE 1
Composition of salt containing solutions

| Name | Composition |
|---|---|
| Formulation of | Sodium acetate (2.5 ml, 7.6M), ethanol (500 μl), benzyl |
| invention | alcohol (125 μl) and PEG-400 (0.5883 g), pyrrolidine |
| | (0.2M, 169 mg) |
| Penetration | Potassium acetate (2.5 ml, 7.6M), ethanol (500 μl), |
| Enhancers | benzyl alcohol (125 μl) and PEG-400 (0.5883 g), |
| (F-PEs) | pyrrolidine (0.2M, 169 mg) |
| Supercooled | Sodium acetate solution (2.5 ml, 7.4M), pyrrolidine |
| Heat (F-Heat) | (0.1M, 93 mg) |
| Control | Potassium acetate solution (2.5 ml, 7.4M), pyrrolidine |
| | (0.1M, 93 mg) |

Sodium acetate was included in the formulation of the invention and also in F-Heat, in order to generate heat. Potassium acetate was used in F-PEs and the Control, as this salt is not capable of generating heat, but allows for comparison in vitro. PEG-400 was added to the formulation of the invention and to F-PEs to ensure that the formulations were homogeneous. In the formulation of the invention, the addition of PEG-400 also ensured that the same heating profiles were obtained from both the formulation of the invention and from F-Heat. The formulation of the invention differed from the other formulations as it provided a combination of enhancers and heat, in comparison to F-PEs, which provided enhancers alone, with no heat, F-Heat, which provided heat, and the Control, which provided neither heat nor enhancers.

### Temperature Profile Measurements

Either a regenerated cellulose or silicone membrane was attached to the lip of a Franz cell donor using ethyl cyanoacrylate glue and tape. The Franz cell was held in a plastic clamp and a surface temperature probe (Hanna Instruments, UK) positioned under the membrane and held in position with a clamp so that the probe was touching the base of the membrane. A 2.5% w/v CMC gel (1 ml) containing lidocaine (15 mg/ml) was added to the donor compartment. The acetate solution was added carefully on top of the gel, and crystallisation was initiated with a seed crystal. The temperature profiles of the four formulations (table 1) were recorded over a period of 30 minutes (n=3). The temperature profile underneath the epidermal sheet was not recorded due to skin availability.

### Drug Transport Studies

Large Franz type diffusion cells (MedPharm Ltd., UK) with a 10 ml receiver capacity and a 2.0 cm² diffusion area mounted with RCM, silicone membrane or human epidermal sheet were used for the drug transport studies. The receiver fluid was phosphate buffered saline (0.15 M, pH 7.3). Franz cells were assembled and sealed using Parafilm® strips. A 2.5% w/v CMC lidocaine saturated (15 mg/ml) gel (1 ml) was used as the drug-containing vehicle and one of the four formulations (Table 1) was added to the gel at the start of the experiment. To ensure the drug was always in the un-ionised form, pyrrolidine was used to adjust the pH so at the lowest point it always remained two pH units above the pKa. Samples (1 ml) for HPLC analysis (LOD=0.23 μg/ml and LOQ=0.75 μg/ml, between 0.05 and 20 μg/ml with a CV<2%) were removed at 0, 5, 10, 15, 20, 25 and 30 minutes from the receiver fluid. After sampling 1 ml of pre-warmed receiver fluid was replaced and corrected for. Studies were conducted in a temperature controlled vivarium (Boddy and Ridewood, UK) with a starting membrane temperature of −32° C. (measured using a surface probe and thermocouple, Hanna Instruments, UK) (n=5).

### Results

### Thermal Profiles

The temperature profiles of the acetate solutions when applied to the CMC drug-loaded gels on the cellulose membrane rose rapidly to a maximum within the first minute (FIG. 1). The maximum membrane temperature for Formulation of the present invention and F-Heat was not statistically different (p>0.05, ANOVA) at 40.8∓0.4° C. and 41.7±0.9° C. respectively. The temperature of the membrane when F-PEs or the control, i.e. non-heat-generating solutions, were applied was 20.8±0.1° C. and 20.7±0.1° C. respectively, which was below the initial experimental testing temperature (˜32° C. to represent the surface of the skin).

Temperature profiles of the acetate solutions measured when applied to a silicone membrane showed an identical profile to the cellulose experiments (FIG. 2). The maximum temperature for the formulation of the invention was 42.1±0.3° C. whilst the maximum temperature of F-Heat was 41.5±0.9° C. The temperature recorded for the non-heat-generating systems over the 30 minute period was ca. 21° C.

### Drug Transport Studies

### Formulation Release

Regenerated cellulose was employed to model the effect of heat on lidocaine release from the formulations. The release profile of lidocaine base through regenerated cellulose membrane from a gel with the application of any of the formulations was relatively linear over the 30 minute time period of the experiment (FIG. 3). The release from the formulation of the invention and F-PEs were both significantly greater (t-test, p<0.05) than the control.

The enhancement ratio of lidocaine release (Table 2) from F-Heat and the formulation of the invention was greatest at T₅ compared to the control although, at every time point, an enhancement was observed. The F-Heat and formulation of the invention had the highest release compared to the control with a 2.2 fold enhancement from both formulations at their maxima. There was no significant difference in release from F-Heat and the formulation of the invention at any time point (t-test, p>0.05). During the experimental period the release from the formulation containing the penetration enhancers alone (F-PEs) was either equal to or significantly lower compared to the control (t-test, p<0.05) (Table 2).

TABLE 2
Cumulative amount of lidocaine (μg/cm²) released through regenerated cellulose
membrane from a lidocaine gel donor (pH adjusted) and one of the four formulations over the
30 minute experimental period. The enhancement ratios refer to the enhancement in
cumulative amount from the test formulations compared to the control.

| | | | | | | Formulation | |
|---|---|---|---|---|---|---|---|
| | Control | F-Heat | | F-PEs | | of the | |
| Time/ | Mean ± | Mean ± | ER ± | Mean ± | | invention | |
| min | SD | SD | SD | SD | ER ± SD | Mean ± SD | ER ± SD |
| 0 | 0 | 0 | x | 0 | X | 0 | x |
| 5 | 3.98 ± 0.29 | 8.80 ± 0.84 | 2.2 ± 0.3 | 2.89 ± 0.57 | 0.7 ± 0.2 | 8.88 ± 0.64 | 2.2 ± 0.2 |
| 10 | 6.75 ± 1.25 | 12.80 ± 2.04 | 1.9 ± 0.5 | 4.81 ± 0.91 | 0.7 ± 0.2 | 11.90 ± 0.54 | 1.8 ± 0.3 |
| 15 | 7.95 ± 1.15 | 15.93 ± 2.83 | 2.0 ± 0.5 | 6.46 ± 0.83 | 0.8 ± 0.2 | 13.81 ± 0.60 | 1.7 ± 0.3 |
| 20 | 9.90 ± 1.05 | 18.82 ± 3.40 | 1.9 ± 0.4 | 8.53 ± 1.09 | 0.9 ± 0.1 | 15.67 ± 0.57 | 1.6 ± 0.2 |
| 25 | 11.98 ± 1.06 | 20.86 ± 4.38 | 1.7 ± 0.4 | 10.47 ± 1.08 | 0.9 ± 0.1 | 17.32 ± 1.04 | 1.4 ± 0.2 |
| 30 | 14.16 ± 1.24 | 22.90 ± 5.07 | 1.6 ± 0.4 | 12.03 ± 1.19 | 0.8 ± 0.1 | 19.28 ± 1.27 | 1.4 ± 0.1 |

### Stratum Corneum (SC) Model (Permeation Without Barrier Alteration)

Silicone membrane is commonly used to model the properties of the SC. In this work, it was employed to demonstrate the effect each of the four formulations had on permeation without introducing the effects of barrier modification. Similar to the release studies over the experimental period, permeation through silicone membrane was relatively linear (FIG. 4).

Each of the three test formulations had significantly greater permeation compared to the control (t-test, p<0.05). Formulation of the present invention had the highest permeation through silicone membrane.

The enhancement ratios (Table 3) illustrated the largest increase in permeation occurred at T₁₅ in comparison to the control. The application of all three test formulations resulted in a quicker time to appearance of lidocaine in comparison to the control with F-Heat showing lidocaine permeation at T₁₀ and F-PEs and the formulation of the invention at T₅, with the formulation of the invention having twice the amount of lidocaine permeated, compared to F-PEs, at that time.

TABLE 3
Cumulative amount of lidocaine (μg/cm²) permeated through silicone
membrane from a lidocaine gel donor (pH adjusted) and one of the four formulations over the
30 minute experimental period. The enhancement ratios refer to the enhancement in
cumulative amount from the test formulations compared to the control.

| | | | | | | Formulation | |
|---|---|---|---|---|---|---|---|
| | Control | F-Heat | | F-PEs | | of the | |
| Time/ | Mean ± | Mean ± | ER ± | Mean ± | | invention | |
| min | SD | SD | SD | SD | ER ± SD | Mean ± SD | ER ± SD |
| 0 | 0 | 0 | x | 0 | X | 0 | x |
| 5 | 0 | 0 | x | 4.20 ± 2.49 | X | 9.21 ± 3.10 | x |
| 10 | 0 | 4.63 ± 2.14 | x | 17.41 ± 5.22 | X | 30.27 ± 5.14 | x |
| 15 | 1.09 ± 0.74 | 12.76 ± 2.77 | 11.7 ± 8.3 | 29.40 ± 7.53 | 26.9 ± 19.6 | 47.17 ± 7.65 | 43.2 ± 30.2 |
| 20 | 3.00 ± 1.25 | 20.64 ± 3.01 | 6.9 ± 3.0 | 40.52 ± 8.34 | 13.5 ± 6.3 | 62.48 ± 10.19 | 20.8 ± 9.3 |
| 25 | 5.26 ± 1.59 | 27.61 ± 2.74 | 5.3 ± 1.7 | 50.87 ± 9.24 | 9.7 ± 3.4 | 75.01 ± 12.16 | 14.3 ± 4.9 |
| 30 | 7.79 ± 1.77 | 33.67 ± 2.62 | 4.3 ± 1.0 | 58.14 ± 9.81 | 7.5 ± 2.1 | 86.08 ± 14.09 | 11.1 ± 3.1 |

### Epidermal Transport

Epidermal sheet was employed as the final barrier, as the largest ex viva effect of heat is expected to be as a result of barrier alteration, by inducing lipid transitions. The permeation of lidocaine through human epidermal sheet (FIG. 5) showed similar linear permeation profiles compared to the permeation of lidocaine through silicone membrane.

Each formulation resulted in a significant increase (p<0.05, t-test) in permeation of lidocaine through the human epidermal sheet compared to the control (0.16±0.13 μg/cm²). After 10 minutes a 2.7-fold increase was observed using F-Heat (0.43±0.03 μg/cm²), a 7.5-fold increase was observed using F-PEs (1.22±0.58 μg/cm²) but almost a 75-fold increase in permeation was observed when the formulation of the invention was used (11.93±5.79 μg/cm²) (FIG. 6).

The enhancement ratios (Table 4) have large variations due to the permeation of lidocaine from the control formulation between T₀ and T₁₀ being below the LOD, which results in a high standard deviation which is used to calculate the enhancement ratios.

TABLE 4
Cumulative amount of lidocaine (μg/cm²) permeated through human epidermal
sheet from a lidocaine gel donor (pH adjusted) and one of the four formulations over the 30
minute experimental period. The enhancement ratios refer to the enhancement in cumulative
amount from the test formulations compared to the control.

| | | | | | | Formulation | |
|---|---|---|---|---|---|---|---|
| | Control | F-Heat | | F-PEs | | of the | |
| Time/ | Mean ± | Mean ± | ER ± | Mean ± | | invention | |
| min | SD | SD | SD | SD | ER ± SD | Mean ± SD | ER ± SD |
| 0 | 0 | 0 | X | 0 | X | 0 | x |
| 5 | 0.06 ± 0.01 | 0.39 ± 0.03 | 6.4 ± 0.8 | 0.28 ± 0.03 | 4.6 ± 0.7 | 0.81 ± 0.32 | 13.1 ± 5.3 |
| 10 | 0.16 ± 0.13 | 0.43 ± 0.03 | 2.8 ± 2.2 | 1.22 ± 0.58 | 7.7 ± 7.2 | 11.93 ± 5.79 | 75.7 ± 71.0 |
| 15 | 0.62 ± 0.81 | 1.31 ± 0.62 | 2.1 ± 2.9 | 2.76 ± 1.13 | 4.4 ± 6.0 | 33.60 ± 3.85 | 54.0 ± 70.3 |
| 20 | 0.68 ± 0.54 | 1.07 ± 0.33 | 1.6 ± 1.4 | 4.18 ± 0.93 | 6.2 ± 5.2 | 48.96 ± 14.28 | 72.4 ± 62.0 |
| 25 | 0.79 ± 0.65 | 2.11 ± 1.62 | 2.7 ± 3.0 | 5.84 ± 0.89 | 7.4 ± 6.1 | 68.68 ± 23.14 | 86.7 ± 76.6 |
| 30 | 0.63 ± 0.61 | 2.21 ± 1.13 | 3.5 ± 3.8 | 8.25 ± 2.16 | 13.0 ± 13.0 | 88.80 ± 35.59 | 140.4 ± 145.7 |

The application of the formulation of the invention resulted in a significant increase (t-test, p<0.05) in permeation compared to the control through human epidermal sheet. This was believed to be due to the simultaneous action of the excipients within the formulation and heat increase the fluidity of the SC lipids facilitating drug transport across the skin.

Attenuated total reflection Fourier transform infrared (ATR-FTIR) was used to confirm the effect of the formulations on the epidermal structure. Points of interest from acquired spectra were the —CH₂ stretching frequency (˜2850 cm⁻¹) where fluidity of SC lipids can be monitored by changes in the degree of organisation of the lipid acyl chains which results in a shift in wave number (Harrison et al., 1996). Each treatment was applied to the surface of the epidermal sheet for 120 seconds. Afterwards the epidermal sheet was scanned between 4000-550 cm⁻¹ with a spectral resolution of 2 cm⁻¹. A lack of treatment of the epidermal sheet was considered to be equivalent to the control, while F-PEs and the formulation of the invention were applied without either PEG-400 or benzyl alcohol due to peak interference. F-Heat was applied as normal. In addition, no gel was applied to underneath the formulations and pyrrolidine was removed from all formulations due to peak interference. Following collection spectra were studied after automatic baseline correction and smoothing in Brüker Optics, Opus version 5.5.

The resulting spectra showed an increase in wave number of the —CH₂ symmetric stretching frequency (˜2850 cm⁻¹), suggesting increased lipid fluidity (FIG. 7). The magnitude of change in wave number of the −CH₂ symmetric stretching frequency was greatest with the formulation of the invention (+2.95 cm⁻¹)>F-PEs (+0.77 cm⁻¹)>F-Heat (+0.40 cm⁻¹)>control. This suggested that an increase in lipid fluidity was the reason for increased drug permeation.

### Conclusions

Release modelled using RCM and permeation through silicone membrane and human epidermal sheet increased significantly when the formulation of the invention was applied compared to the control. Release of lidocaine from the formulations and permeation were higher when the formulations contained alcohols. Permeation through silicone membrane and epidermal sheet was significantly higher when the formulation contained alcohols and generated heat (the formulation of the invention). The greatest enhancement in permeation compared to the control was observed when the formulation of the invention was applied to human epidermal sheet. The enhancement in permeation observed with the formulation of the invention through epidermal sheet appeared to be as a result of increased barrier fluidity. The simultaneous application of alcohols and heat was seen to significantly enhance the release and permeation in both model membranes and ex vivo barriers. The significantly greater enhancements observed within the permeation through ex vivo epidermal sheet was due to the synergistic effect of both heat and alcohol increasing the mobility of the barrier.

### Example 2

### Formulation of the Invention Dual Spray Systems

In addition to the more traditional semi-solid formulations illustrated in Example 1, a dual spray system was used to demonstrate that exothermic crystallisation can be initiated via expulsion from a canister and which results in increased permeation compared to a non-heat-generating control.

### Preparation of Supercooled Spray Systems

Formulations using similar components to the semi-solid formulations described earlier were prepared in two canisters (Table 5). A CMC gel with ethanol and lidocaine base was added into Pump A, whilst the supercooled salts, prepared by heating an amount of salt in water at 80° C. were added to Pump B. Sodium acetate was used as the exothermic salt and potassium acetate was used as a non-heat-generating mimetic control. Each pump contained pyrrolidine to ensure that lidocaine remained un-ionised throughout the experiment.

TABLE 5
Composition of the pumps for the
dual spray (DS) delivery systems.

| System | Pump A | | Pump B | |
|---|---|---|---|---|
| Formulation | Water | 10 ml | Sodium acetate | 2.5 ml |
| of the | Ethanol | 2.5 ml | solution | |
| invention DS | CMC (1.75% w/w) | 210 mg | (8.55M)* | |
| | Lidocaine base | 100 mg | Pyrrolidine | 50 mg |
| | Pyrrolidine | 25 mg | 0.5M | |
| Control DS | Water | 10 ml | Potassium acetate | 2.5 ml |
| | Ethanol | 2.5 ml | solution | |
| | CMC (1.75% w/w) | 210 mg | (8.55M)* | |
| | Lidocaine base | 100 mg | Pyrrolidine | 50 mg |
| | Pyrrolidine | 25 mg | 0.5M | |

### Thermal Profile Measurements

A wire temperature probe (Hanna Instruments, UK) was positioned at the base of a glass vial held at a 45° angle by a plastic clamp stand. Pump A was carefully sprayed into the compartment 12 times; at the same time Pump B was sprayed into the same compartment with the sprays from each pump combining before they reached the base of the vial. The temperature profiles were recorded over a 30 minute period (n=3).

The temperature profiles of the dual spray formulations were assessed at room temperature (FIG. 8). The maximum temperature for the formulation of the invention DS was 46.0±0.5° C. which was reached within 45 seconds, and remained elevated (above 32° C.) for ca. 525 seconds. The Control DS reached a maximum temperature of 21.6±0.2° C. in approximately 120 seconds. The temperature of the Control DS was always lower than the surface temperature of skin.

### Drug Transport Studies

Large Franz type diffusion cells (MedPharm Ltd., UK) with a 10 ml receiver capacity and a 2.0 cm² diffusion area were mounted with silicone membrane. The receiver fluid was phosphate buffered saline (0.15 M, pH 7.3). Franz cells were assembled then sealed using Parafilm® strips. The formulation was applied onto the surface of the Franz cell using the two pump sprays, each spray delivered 100 μl of formulation. Pump A was sprayed 12 times (delivering ca. 1.2 ml) and Pump B was sprayed 13 times (delivering ca. 1.3 ml). Samples (1 ml) were removed for HPLC analysis at 0, 5, 10, 15, 20, 25 and 30 minutes. Studies were conducted inside a temperature controlled vivarium (Boddy and Ridewood, UK) to ensure the starting temperature of the membrane was ˜32° C.

The permeation profile of Control DS was linear over the 30 minute period. The formulation of the invention exhibited substantial and linear, permeation, although permeation began to slow down toward the end of the 30 minutes (FIG. 9). The permeation of lidocaine from the formulation of the invention DS was significantly higher (t-test, p<0.05) at every time point in comparison to Control DS.

The enhancement ratios (Table 6) illustrated the effect that heat produced from the formulation of the invention DS had on permeation in comparison to Control DS. The largest effect was observed at T₅, although enhancements were noted at every time point.

TABLE 6
Cumulative amount of lidocaine (μg/cm²) permeated through
silicone membrane when delivered from two dual spray systems.

| | | Formulation of | |
|---|---|---|---|
| | Control DS | the invention DS | |
| Time/min | Mean ± SD | Mean ± SD | ER ± SD |
| 0 | 0 | 0 | x |
| 5 | 1.4 ± 0.9 | 12.0 ± 1.8 | 8.8 ± 5.9 |
| 10 | 4.3 ± 2.6 | 32.4 ± 1.5 | 7.6 ± 4.7 |
| 15 | 7.5 ± 4.0 | 48.9 ± 3.5 | 6.6 ± 3.5 |
| 20 | 11.0 ± 4.4 | 60.9 ± 6.1 | 5.5 ± 2.3 |
| 25 | 13.4 ± 4.4 | 70.1 ± 7.6 | 5.2 ± 1.8 |
| 30 | 16.8 ± 5.3 | 80.6 ± 7.0 | 4.8 ± 1.6 |

### Example 3

The Effect of Ethanol, Benzyl Alcohol and PEG-400 on Permeation of Lidocaine Base through Silicone Membrane

### Objective

To compare the effects of excipients of the invention on permeation, by elimination. In this Example, the formulation designated Test Formulation was constituted as the formulation designated ‘Formulation of the invention’ in Example 1.

### Method

Stock solutions of the supercooled salts were prepared as before. The following formulations (Table 7) were then tested to see if they enhanced lidocaine permeation compared to the control.

TABLE 7
Composition of salt containing solutions.

| Name | Composition |
|---|---|
| Test Formulation | Sodium acetate (2.5 ml, 7.6M), ethanol (500 μl), |
| | benzyl alcohol (125 μl) and PEG-400 (0.5883 g), |
| | pyrrolidine (0.2M, 169 mg) |
| Heat plus benzyl | Sodium acetate solution (7.6M, 2.5 ml), ethanol |
| alcohol and ethanol | (500 μl), benzyl alcohol (125 μl), pyrrolidine |
| (F-ΔAlcohol) | (0.2M, 169 mg) |
| Heat plus EtOH | (7.6M, 2.5 ml), ethanol (500 μl), pyrrolidine |
| (F-ΔEtOH) | (0.2M, 169 mg) |
| Supercooled Heat | Sodium acetate solution (2.5 ml, 7.4M), pyrrolidine |
| (F-Heat) | (0.1M, 93 mg) |

### Results

The permeation of lidocaine from all of the formulations was relatively linear over the 30 minute time period (FIG. 10).

The permeation profiles clearly indicate that heat with any additional excipients results in a significantly higher permeation (t-test, P<0.05) compared to heat alone.

The enhancement ratios (Table 8) illustrated the largest increase in permeation occurred at T₁₀ in comparison to the F-Heat, however the application of all three test formulation resulted in a quicker time to appearance of lidocaine (T₅ instead of T₁₀) in comparison to F-Heat.

TABLE 8
Cumulative amount of lidocaine (μg/cm²) permeated through silicone membrane
from a lidocaine gel donor (pH adjusted) and one of the four formulations over the 30 minute
experimental period. The enhancement ratios refer to the enhancement in cumulative amount
from the test formulations compared to the F-Heat (heat alone).

| | F-Heat | Test | | | | | |
|---|---|---|---|---|---|---|---|
| Time/ | Mean ± | Formulation | ER ± | F-ΔAlcohol | ER ± | F-ΔEtOH | ER ± |
| min | SD | Mean ± SD | SD | Mean ± SD | SD | Mean ± SD | SD |
| 0 | 0 | 0 | 0 | 0 | 0 | 0 | 0 |
| 5 | 0 | 9.21 ± 3.10 | 0 | 20.88 ± 5.44 | 0 | 34.06 ± 9.31 | 0 |
| 10 | 4.63 ± 2.14 | 30.27 ± 5.14 | 6.5 ± 3.2 | 52.24 ± 7.77 | 11.3 ± 5.5 | 65.19 ± 6.46 | 14.1 ± 7.4 |
| 15 | 12.76 ± 2.77 | 47.17 ± 7.65 | 3.7 ± 1.0 | 71.70 ± 6.58 | 5.6 ± 1.3 | 89.24 ± 15.85 | 7.0 ± 2.0 |
| 20 | 20.64 ± 3.01 | 62.48 ± 10.19 | 3.0 ± 0.7 | 82.39 ± 7.82 | 4.0 ± 0.7 | 108.26 ± 17.28 | 5.3 ± 1.1 |
| 25 | 27.61 ± 2.74 | 75.01 ± 12.16 | 2.7 ± 0.5 | 98.80 ± 10.14 | 3.6 ± 0.5 | 130.93 ± 25.49 | 4.7 ± 1.0 |
| 30 | 33.67 ± 2.62 | 86.08 ± 14.09 | 2.6 ± 0.5 | 112.70 ± 13.68 | 3.4 ± 0.5 | 147.81 ± 28.06 | 4.4 ± 0.9 |

In addition, the permeation of lidocaine from F-ΔEtOH was significantly higher at all time points compared to Test Formulation. There was no statistical difference between the permeation of lidocaine from F-ΔEtOH and F-ΔAlcohol, but F-ΔEtOH was higher. Removal of PEG-400 from the formulations appeared to result in increased lidocaine permeation through the membrane (when comparing to Test Formulation, although the differences were not significant).

### Comparative Example

### Alternative Heat Source, Active Pharmaceutical Ingredients (APIs), and Penetration Enhancers

In order to demonstrate that the supercooled salt solution is essential, and that the higher rates of transdermal flux obtained with supercooled salt solutions are not simply as a result of increased local temperature, a variety of API test formulations was produced. Three active pharmaceutical ingredients were selected, based on their different physicochemical properties.
Sildenafil base (http://pubchem.ncbi.nlm.nih.gov/)—(experimentally determined)<0.83 mg/ml, MW 474.57, (calculated) Log P 1.5
Diclofenac diethyl amine (DDEA) (A. Fini et al., 1996. Factors governing the dissolution of diclofenac salts, European Journal of Pharmaceutical Sciences, vol. 4 (4), 231-238; V. Tantishaiyakul, 2004. Prediction of aqueous solubility of organic salts of diclofenac using PLS and molecular modelling. International Journal of Pharmaceutics, 275, 133-139)—4.1 mg/ml, MW 36929, (calculated) Log P 0.54
Acyclovir (http://www.drugbank.ca/) 1.62 mg/ml, MW 225.20, Log P −1.56

The following penetration enhancers were selected for incorporation into the test formulations, based on the solubility of the API within the enhancer. Levels of penetration enhancer were selected in accordance with FDA guidelines for topical application. Enhancers selected were:
benzyl alcohol
isopropyl alcohol
menthol
ethanol
Transcutol P (diethylene glycol monoethyl ether)
Arlasolve (dimethyl isosorbide)

PEG-400, propylene glycol and water were employed as solubilising agents for the API in the test formulations.

### Methods

### Analytical Assays

### Sildenafil Base Assay

Sildenafil base was analysed using HPLC, and included a Waters 2695 separations (Multi system of Column heater, sample heater, and pump), and a Waters 996 Photo-diode array detector. The temperature of the stationary phase and samples were maintained at 25.0±5° C. and 5.0±2° C., respectively. The stationary phase employed was a Sunfire C8 (Waters, USA) column (100×4.6 mm 3 μm). Mobile phase A, 0.2M ammonium acetate buffer pH 7.00, and Mobile phase B, 100% acetonitrile were used with an isocratic flow 50:50 composition, at a flow rate of 1.0 mL/min. An injection volume of 20 μL with a run time of 20 min was used. The averaged retention time of sildenafil base was 5 min at a detection wavelength of 224 nm. Calibration curves were constructed from a series of standards prepared by dilution along with separately prepared quality controls. Standards and QC's were diluted using receiver fluid (20% ethanol in citric acid-disodium hydrogen phosphate buffer in deionised water adjusted to pH 6.5). Data were recorded and analysed using EmPower Pro2 Software (2002). The LOD and LOQ between 0.50 and 100.00 μg/ml were 1.39 μg/ml and 4.22 μg/ml, respectively.

### DDEA Assay

DDEA was analysed using HPLC, and included a Waters 2695 separations (Multi system of Column heater, sample heater, and pump), and a Waters 996 Photo-diode array detector. The temperature of the column and samples were maintained at 30±2° C. and 5.0±2° C., respectively. A Gemini™ C-18 (Phenomenex, USA) column (150 mm×4.6 mm 5 μm particle size) with a guard column (Phenomenex USA, C-18 4.0×3.0 mm) was employed as the stationary phase. The mobile phase was two part; mobile phase A, 20 mM ammonium formate (in deionised water) buffer pH 2.5, and Mobile phase B, 20 mM ammonium formate in acetonitrile, run using an isocratic flow of 35:65 composition with a flow rate of 1.0 mL/min. Samples were run for 20 minutes with an injection volume of 5 μL, the average retention time of DDEA was 10 min at a detection wavelength of 280 nm. Calibration curves were constructed from a series of standards prepared by serial dilution in conjunction with separately prepared quality controls. Both standards and QC's were diluted with receiver fluid (20% ethanol in phosphate buffered saline). Data were recorded and analysed using EmPower Pro2 Software (2002). The LOD and LOQ between 0.05 and 1.00 μg/ml were 0.23 μg/ml and 0.69 μg/ml, respectively.

### Acyclovir Assay

Acyclovir was analysed using a Waters 2695 separations (Multi system of Column heater, sample heater, and pump), and a Waters 2487 Dual λ absorbance detector. The temperature of the column and samples were maintained at 25.0±5° C. and 20.0±2° C., respectively. A HyperCarb (porous graphitic carbon, Thermo scientific, USA) column (100×4.6 mm 3 μm) and a universal uniguard (10.00×4.00 mm guard, Thermo scientific, USA) was the stationary phase. An isocratic flow 80:20 composition with a flow rate of 1.0 mL/min of two mobile phases; mobile phase A, 1% TFA in 90% deionised water (18.2 MΩ·cm) 10% acetonitrile and mobile phase B 1% TFA in 90% acetonitrile 10% deionised water (18.2 MΩ·cm) was employed. The sample injection volume was 30 μL. Acyclovir quantification was determined at two wavelengths, 285 nm and 380 nm with a retention time between 5-10 min. The run time for standard was 10 min, all other samples were run for 20 min. Calibration curves were constructed from a set of serial diluted standard along with separately prepared quality control. Both standards and QC's were diluted with receiver fluid (Phosphate buffered saline). Data were analysed using EmPower Prot Software (2002). The LOD and LOQ between 0.05 and 50 μg/ml were 0.35 μg/ml and 1.07 μg/ml, respectively.

### API In Vitro Permeation Studies

### Preparation of Test Solutions

Test solutions were prepared by saturating the solvent system with the API at the membrane temperature (32 or 45° C.) for approximately 12 h. The quantity of API required for each system was predicted using the provisional API solubility tests. It was ensured that a fine precipitate was observed in each test solution prior to dosing in the in vitro permeation experiments.

### Sildenafil Base Test Solutions

| | SB-SS | SB-SS1 | SB-SS2 |
|---|---|---|---|
| Excipient | % w/w | % w/w | % w/w |
| Water | 100.00 | 98.00 | 88.00 |
| Benzyl alcohol | | 2.00 | 2.00 |
| Arlasolve | | | 10.00 |
| TOTAL | 100.00 | 100.00 | 100.00 |

### DDEA Test Solutions

| | DDEA-SS | DDEA-SS1 | DDEA-SS2 |
|---|---|---|---|
| Excipient | % w/w | % w/w | % w/w |
| PEG-400 | 100.00 | 90.00 | 89.00 |
| IPA | | 10.00 | 10.00 |
| Menthol | | | 1.00 |
| TOTAL | 100.00 | 100.00 | 100.00 |

### Acyclovir Test Solutions

| | ACV-SS | ACV-SS1 | ACV-SS2 |
|---|---|---|---|
| Excipient | % w/w | % w/w | % w/w |
| Water | 80.00 | 66.00 | 52.00 |
| Propylene glycol | 20.00 | 16.50 | 13.00 |
| Ethanol | | 17.50 | 17.50 |
| Transcutol P | | | 17.50 |
| TOTAL | 100.00 | 100.00 | 100.00 |

### Preparation of Epidermal Sheet

Human epidermal membrane was prepared from skin post cosmetic reduction surgery (abdominoplasty). Full thickness was defrosted at ambient temperature until malleable. The subcutaneous fat was removed mechanically by blunt dissection. Upon removal of the fat, skin was immersed in hot deionised water (60±3° C.) for 45 s. The epidermal membrane (comprising the Stratum corneum and epidermis) was removed from the underlying dermis using a gloved finger and the dermis was discarded. The epidermal membrane was then floated (Stratum corneum side up) in deionised water onto filter paper. Excess water was removed from the surface and the tissue was stored until required.

### In Vitro Permeation Investigation

Individually calibrated unjacketed small Franz type diffusion cells were employed. Each cell had an average surface area approximately 0.60 cm² and a volume of approximately 2.0 mL Studies were conducted at two temperatures, so the surface temperature of the skin would be maintained at 32° C. and 45° C.

A total of 58 cells were prepared, with nine active formulations, n=3 at each temperature and one blank cell, with no formulation applied, per API. The integrity of the epidermal membrane was assessed visually prior to dosing the cells. The lower receptor chamber was filled with respective receiver fluid (ACV: phosphate buffered saline solution, SB: 20% ethanol in disodium hydrogen phosphate buffer pH 7.00 and DDEA: 20% ethanol in phosphate buffered saline solution). Each cell was then inverted and observed for leaks. If no leaks were observed the cells were used, if leaks were observed the cell was remounted.

Cells were placed in the water bath and allowed to equilibrate for 30 min prior to dosing. A 200 μL dose of each formulation was applied to 6 cells (3 with a membrane temperature of 32° C. and 3 with a membrane temperature of 45° C.). Each cell was dosed using a pre-calibrated positive displacement pipette. Following application of the test formulation, receiver fluid (200 μL) was removed from the sampling arm at 0, 30 min, 1, 2, 4, 6, 24, 30 and 48 h time points. After each sample was removed, an equal volume of pre-warmed receiver fluid was replaced. Samples were analysed using HPLC using the corresponding API's analytical method.

### Results—API In Vitro Permeation Studies

The permeation of each test formulation was assessed at membrane temperatures of 32 and 45° C. The permeation from each test formulation was measured in triplicate at each temperature. Cells were rejected from the study on the basis of HPLC inaccuracies and leakage.

### In Vitro Permeation of Sildenafil Base Through Epidermal Sheet

Sildenafil permeation through epidermal sheet was increased with both heat and penetration enhancers independently (Table 9).
A large increase in sildenafil permeation was observed at 24 hours with an elevated temperature and benzyl alcohol present in the formulation (SS1 at 45° C., mean value 25.99 μg/cm²) in comparison to heat alone (SS at 45° C., mean value 8.77 μg/cm²), penetration enhancers alone (SS1 at 32° C., mean value 7.42 μg/cm²) and neither heat or penetration enhancers (SS at 32° C., mean value 4.34 μg/cm²) (FIG. 11).
An increase in sildenafil permeation when both arlasolve and benzyl alcohol were in the formulation and heat was applied (SS2 at 45° C., mean value 8.24 μg/cm²) was observed in comparison to no heat and no penetration enhancers (SS at 32° C., mean value 4.34 μg/cm²) but the increase was not as large as the penetration enhancers alone (SS2 at 32° C., mean value 11.04 μg/cm²) (FIG. 11).

Table 9 shows the data obtained for mean cumulative permeation of sildenafil from the three saturated test formulations at two different membrane temperatures (32 and 45° C.). Enhancement ratio (ER) refers to the enhancement compared to SB-SS at 32° C.

TABLE 9

| | Formulation | | | | | | | | | | |
|---|---|---|---|---|---|---|---|---|---|---|---|
| | SB-SS | SB-SS1 | | SB-SS2 | | SB-SS | | SB-SS1 | | SB-SS2 | |
| | 32° C. | 32° C. | | 32° C. | | 45° C. | | 45° C. | | 45° C. | |
| | Mean API | Mean API | | Mean API | | Mean API | | Mean API | | Mean API | |
| | permeated | permeated | Mean | permeated | Mean | permeated | Mean | permeated | Mean | permeated | Mean |
| Time (h) | (μg/cm²) | (μg/cm²) | ER | (μg/cm²) | ER | (μg/cm²) | ER | (μg/cm²) | ER | (μg/cm²) | ER |
| 0.5 | 2.33* | 1.96* | 0.84 | 1.82* | 0.78 | 1.70* | 0.73 | 2.71* | 1.16 | 2.32* | 1.00 |
| 1.0 | 4.04* | 4.28 | 1.06 | 2.01* | 0.50 | 2.01* | 0.50 | 3.94* | 0.97 | 3.28* | 0.81 |
| 2.0 | 1.93* | 3.23* | 1.67 | 2.89* | 1.50 | 2.32* | 1.20 | 8.64 | 4.47 | 3.95* | 2.04 |
| 4.0 | 3.03* | 3.70* | 1.22 | 3.69* | 1.22 | 3.82* | 1.26 | 11.45 | 3.77 | 5.60 | 1.85 |
| 6.0 | 3.17* | 10.96 | 3.45 | 4.68 | 1.47 | 4.27 | 1.34 | 12.16 | 3.83 | 6.71 | 2.11 |
| 24.0 | 4.34 | 7.42 | 1.71 | 11.04 | 2.55 | 8.77 | 2.02 | 25.99 | 5.99 | 8.24 | 1.90 |
| 30.0 | 4.98 | 8.99 | 1.80 | 12.29 | 2.47 | 10.86 | 2.18 | 26.62 | 5.34 | 8.89 | 1.78 |
| 48.0 | 6.41 | 8.61 | 1.34 | 30.03 | 4.69 | 14.83 | 2.31 | 34.38 | 5.36 | 11.87 | 1.85 |

*denotes a value below the LOQ therefore accuracy cannot be guaranteed, x denotes no API detection.

### In Vitro Permeation of DDEA Through Epidermal Sheet

DDEA permeation from all formulations was comparable for the first 6 hours of the study. At 24 and 30 hours large differences in DDEA permeation were observed, with formulations at a higher membrane temperature exhibiting greater DDEA permeation. In addition, incorporation of penetration enhancers to the system increased DDEA permeation (Table 10, FIG. 12).
An increase in DDEA permeation was observed at 30 hours with an elevated temperature and IPA and menthol present in the formulation (SS2 at 45° C., mean value 3.54 μg/cm²) in comparison to heat alone (SS at 45° C., mean value 1.43 μg/cm²), penetration enhancers alone (SS1 at 32° C., mean value 0.42* μg/cm²) and neither heat or penetration enhancers (SS at 32° C., mean value 0.04* μg/cm²) (FIG. 13, Table 10, *denotes value below LOQ).

Table 10 provides data relating to mean cumulative permeation of DDEA from the three saturated test formulations at two different membrane temperatures (32 and 45° C.). Enhancement ratio (ER) refers to the enhancement compared to DDEA-SS at 32° C.

TABLE 10

| | Formulation | | | | | | | | | | |
|---|---|---|---|---|---|---|---|---|---|---|---|
| | DDEA-SS | DDEA-SS1 | | DDEA-SS2 | | DDEA-SS | | DDEA-SS1 | | DDEA-SS2 | |
| | 32° C. | 32° C. | | 32° C. | | 45° C. | | 45° C. | | 45° C. | |
| | Mean API | Mean API | | Mean API | | Mean API | | Mean API | | Mean API | |
| Time | permeated | permeated | Mean | permeated | Mean | permeated | Mean | permeated | Mean | permeated | Mean |
| (h) | (μg/cm²) | (μg/cm²) | ER | (μg/cm²) | ER | (μg/cm²) | ER | (μg/cm²) | ER | (μg/cm²) | ER |
| 0.5 | x | x | — | x | — | x | — | 0.00* | — | x | — |
| 1.0 | x | x | — | x | — | 0.01* | — | 0.07* | — | x | — |
| 2.0 | x | x | — | 0.10* | — | x | — | 0.04* | — | 0.05* | — |
| 4.0 | x | 0.01 | — | 0.13* | — | 0.02* | — | 0.06* | — | 0.12* | — |
| 6.0 | x | x | 0.59 | 0.20* | 46.66 | 0.04* | 8.69 | 0.06* | 13.69 | 0.17* | 40.57 |
| 24.0 | 0.01* | 0.08* | 6.08 | 0.34* | 25.67 | 0.80 | 61.08 | 0.39* | 30.07 | 1.30 | 99.58 |
| 30.0 | 0.04* | 0.15* | 3.52 | 0.42* | 10.12 | 1.43 | 34.06 | 1.05 | 25.14 | 3.54 | 84.46 |

### In Vitro Permeation of ACV Through Epidermal Sheet

Synergistic enhancement in acyclovir permeation was observed at 48 hours when transcutol P and ethanol were included in the formulation and heat was applied (SS2 at 45° C., mean value 22.46 μg/cm²) in comparison to the penetration enhancers alone (SS2 at 32° C., mean value 10.90 μg/cm²), heat alone (SS at 45° C., mean value 2.37 μg/cm²) or no heat and no penetration enhancers (SS at 32° C., mean value 0.74* μg/cm²). (FIG. 14, Table 11, *denotes value below LOQ).

Table 12 provides data relating to mean cumulative permeation of acyclovir from the three saturated test formulations at two different membrane temperatures (32 and 45° C.). Enhancement ratio (ER) refers to the enhancement compared to ACV-SS at 32° C.

TABLE 12

| | Formulation | | | | | | | | | | |
|---|---|---|---|---|---|---|---|---|---|---|---|
| | ACV-SS | ACV-SS1 | | ACV-SS2 | | ACV-SS | | ACV-SS1 | | ACV-SS2 | |
| | 32° C. | 32° C. | | 32° C. | | 45° C. | | 45° C. | | 45° C. | |
| | Mean API | Mean API | | Mean API | | Mean API | | Mean API | | Mean API | |
| | permeated | permeated | Mean | permeated | Mean | permeated | Mean | permeated | Mean | permeated | Mean |
| Time (h) | (μg/cm²) | (μg/cm²) | ER | (μg/cm²) | ER | (μg/cm²) | ER | (μg/cm²) | ER | (μg/cm²) | ER |
| 0.5 | x | 0.03* | — | 0.16* | — | x | — | 0.07* | — | 0.19* | — |
| 1.0 | x | 0.11* | — | 0.24* | — | 0.34* | — | 0.18* | — | 0.34* | — |
| 2.0 | x | 0.08* | — | 0.15* | — | 0.71* | — | 0.10* | — | 0.21* | — |
| 4.0 | 0.08* | 0.08* | 0.97 | 0.18* | 2.21 | 0.72* | 8.87 | 0.29* | 3.57 | 0.32* | 3.94 |
| 6.0 | 0.05* | 0.11* | 2.33 | 0.34* | 6.99 | 0.73* | 15.16 | 0.12* | 2.47 | 0.24* | 4.94 |
| 24.0 | 0.37* | 0.45* | 1.22 | 3.94 | 10.60 | 0.44* | 1.19 | 0.64* | 1.73 | 3.94 | 10.59 |
| 30.0 | 0.61* | 0.53* | 0.87 | 5.16 | 8.48 | 0.78* | 1.29 | 1.49 | 2.45 | 9.12 | 14.98 |
| 48.0 | 0.74* | 1.49 | 2.02 | 10.90 | 14.75 | 2.37 | 3.21 | 2.64 | 3.57 | 22.46 | 30.39 |

*denotes a value below the LOQ therefore accuracy cannot be guaranteed, x denotes no API detection.

### Conclusions

Elevated, continuous heat improves API uptake, but only after a number of hours has elapsed.

This Example demonstrates that it is not simply the elevated temperature that is responsible for higher transdermal flux.

### Abbreviations

Abbreviation Definition
ACV Acyclovir
API Active pharmaceutical ingredient
DFA Diclofenac Free Acid
DMI Arlasolve Dimethyl Iso-sorbide
DUR₃₂ Duration above 32° C.
IPA 2-Propanol
LOD Limit of detection
LOQ Limit of quantification
PEG-400 Polyethylene glycol-400
PG Propylene glycol
SA Sodium Acetate
SC Stratum corneum
SDC Sildenafil Citrate
SS Solvent system
ST Sodium Thiosulphate
TP Transcutol P
tₘₐₓ Time to maximum temperature
Tₘₐₓ Maximum temperature

Examples 1 to 3, above, demonstrate the principal of the combination of heat and permeation enhancer in the goal of enhancing transdermal drug delivery. In the following Examples, heat may be supplied by other means, but operates on the concept of pre-heating the skin to ≧40° C., more preferably 42° C. or higher, and preferably ˜45° C., before allowing the skin temperature to return to ˜32° C. It is shown that raising the temperature in this way for 10 minutes or more, preferably around 15 minutes, has a significant effect on the formulations and methods of the present invention, sometimes as much as 24 hours or more later.

The following Examples used three model compounds; sildenafil citrate, acyclovir and diclofenac free acid.

### Methods

### Assessment of Thermogenic Materials

### Heat of Hydration

Although there is an extensive list of compounds that can generate heat via hydration, the anhydrous salts calcium chloride (CaCl₂) and magnesium sulphate (MgSO₄) were selected for illustrative purposes and their heat generating properties were characterised. The two anhydrous salts were investigated at three different levels (0.5, 1.0 and 2.0 g). Each level of salt was weighed into a vial and a volume of water (1.0 or 2.0 mL) was added using a Gilson pipette. The temperature was recorded using a data logger with a baseline temperature of 32° C., to mimic the skins surface temperature.

### Enthalpy of Polymerisation

The enthalpy of polymerisation of bisphenol-A (BPA) and epichlorohydrin (ECH) was characterised in terms of heat generation. ECH and BPA were dissolved in solutions of potassium hydroxide (40%), added to a glass vial in the ratio of 10:1 and mixed for one hour. Upon completion of the reaction an epoxy resin was obtained. The temperature was recorded using a data logger with a baseline temperature of 32° C., to mimic the skins surface temperature.

### Exothermic Supercooled Salt Solutions

### Temperature Profiling of Exothermic Salts

The thermogenic properties of two salts; sodium acetate and sodium thiosulphate were investigated at different saturation levels. Initially, the boundaries of metastable saturation were established. Upon establishment of the metastable region solutions were prepared at different saturation levels and the temperature of 1.0 and 2.5 mL of solution was assessed after the addition of a seed crystal. The solutions were prepared by the addition of salt (10.0 g) with water at 1.0 g intervals. Intervals of water ranged from 5.0 g, to 12.0 g for SA and 7.0 g, to 15.0 g for ST. The salt and water were weighed into a Duran bottle (50 mL) and the solution was heated at ca. 80° C. whilst stirring. Upon formation of a single phase solution, each solution was separated into 1.0/2.0 mL volumes using a syringe and was left to cool. Once cooled the thermogenic properties of each solution was assessed using a data logger.

### Analytical Assays

### Sildenafil Citrate Assay

Sildenafil citrate was analysed using both HPLC and UPLC. The HPLC system was composed of a Waters 2695 Alliance HPLC system, a Waters 996 Photo-diode array (at detection wavelength 224 and 294) and was processed using Waters Empower³ data processing software. A Waters Sunfire C8 3.5 μm was used with a column temperature of 25±5° C. The sample temperature was maintained at 5±2° C. The flow rate was set at 1.0 mL/min with an isocratic method (50:50, mobile phase A: mobile phase B). Mobile Phase A was 0.2 M Ammonium acetate buffer pH 7.0 and Mobile Phase B was 100% ACN. Samples were injected at a volume of 20 μL and a run time of 20 minutes. The average retention time of sildenafil citrate was observed to be at 4 minutes.

The UPLC was composed of a Waters Acquity UPLC System, Waters 996 Photo-diode array detector, Waters binary solvent system and a Waters sample manager. The data was processed with Waters empower³ data processing software. An Acquity UPLC BEH C8 1.7 μm 2.1×100mm column at a column temperature of 40±5° C. was used. Samples detection was at a wavelength of 224 (and an injection volume of 7.5 μL.) The temperature of these samples was 5±2° C. An isocratic method of Mobile Phase A (10 mM Ammonium acetate buffer pH 7.0) and Mobile Phase B (100% ACN) at a flow rate of 0.6 mL/min was used. The run time of the system was 5 minutes with an average retention time of 2.5 minutes and an injection volume of 7.5 μL.

### Acyclovir Assay

Acyclovir was analysed using a Waters 2695 separations (Multi system of Column heater, sample heater, and pump), a Waters 2487 Dual λ absorbance detector. The temperature of the column and samples were maintained at 25.0±5° C. and 20.0±2° C., respectively. A HyperCarb (porous graphitic carbon, Thermo scientific, USA) column (100×4.6 mm 3 μm) and a universal uniguard (10.00×4.00 mm guard, Thermo scientific, USA) was the stationary phase. An isocratic flow 80:20 composition with a flow rate of 1.0 mL/min of two mobile phases; mobile phase A, 1% TFA in 90% deionised water (18.2 MΩ·cm) 10% acetonitrile and mobile phase B 1% TFA in 90% acetonitrile 10% deionised water (18.2 MΩ·cm) was employed. The sample injection volume was 30 μL. Acyclovir quantification was determined at two wavelengths, 285 nm and 380 nm with a retention time between 5-10 min. The run time for standards was 10 min. All other samples were run for 20 min. Calibration curves were constructed from a set of serial diluted standard along with separately prepared quality control. Both standards and QC's were diluted with receiver fluid (Phosphate buffered saline). Data were analysed using EmPower Pro³ Software (2010).

### Diclofenac Assay

Diclofenac was analysed using HPLC that comprised of a Waters 2695 separations (Multi system of Column heater, sample heater, and pump), a Waters 996 Photo-diode array detector. The temperature of the column and samples were maintained at 25±5° C. and 5.0±2° C., respectively. A Symmetry Shield™ C-18 (Waters, USA) column (150 mm×4.6 mm 5 μm particle size) with a guard column (Waters USA, C-18 4.0×3.0 mm) was employed as the stationary phase. The mobile phase was mobile phase A, 50.7% Acetonitrile, 47.8% Water and 1.5% Acetic Acid run using an isocratic flow with a flow rate of 1.5 mL/min. Samples were run for 15 minutes with an injection volume of 75 μL, the average retention time of diclofenac was 7 min at a detection wavelength of 254 nm. Calibration curves were constructed from a series of standards prepared by serial dilution in conjunction with separately prepared quality controls. Both standards and QC's were diluted with receiver fluid (PBS). Data were recorded and analysed using EmPower Pro³ Software (2010).

### Solubility Assessment

Visual solubility of sildenafil citrate, acyclovir and diclofenac was assessed in deionised water, polyethylene glycol 400 (PEG-400), ethanol, isopropyl alcohol, propylene glycol, Transcutol P, phenoxyethanol, diethyl ether, isoamyl alcohol, arlasolve DMI and methanol.

The solubility experiments were performed by weighing 0.5±0.05 g of each excipient into glass vials (28 mL). The API (5.0±0.5 mg) was weighed by difference on a plastic weigh boat and transferred into the corresponding vial. The excipient and API were stirred for approximately 2 h, subsequent aliquots of API were added if the solution had dissolved or further solvent was added if the solution was still saturated. Once saturation was achieved the mg/g API to solvent ratio was recorded.

### API In Vitro Permeation Studies

### Preparation of Donor Solutions

Donor solutions were prepared by saturating the solvent system with the API for approximately 12 h. The quantity of API required for each system was predicted using the provisional API solubility tests. It was ensured that a fine precipitate of API was observed in each donor solution prior to dosing in the in vitro permeation experiments.

### Sildenafil Citrate Donor Solutions

| | System 1 | System 2 | System 3 | System 4 |
|---|---|---|---|---|
| Excipient | % w/w | % w/w | % w/w | % w/w |
| Water | 100.00 | 90.00 | 90.00 | 80.00 |
| MeOH | | 10.00 | | 10.00 |
| Transcutol P | | | 10.00 | 10.00 |
| TOTAL | 100.00 | 100.00 | 100.00 | 100.00 |

### Diclofenac Free Acid Donor Solutions

| | System 1 | System 2 | System 3 | System 4 |
|---|---|---|---|---|
| Excipient | % w/w | % w/w | % w/w | % w/w |
| Water | 20.00 | 18.00 | 18.00 | 18.00 |
| PEG-400 | 75.00 | 72.00 | 72.00 | 72.00 |
| EtOH | | 10.00 | 10.00 | 5.00 |
| DMI/Arlasolve | | | | 5.00 |
| TOTAL | 100.00 | 100.00 | 100.00 | 100.00 |

### Acyclovir Donor Solutions

| | System 1 | System 2 |
|---|---|---|
| Excipient | % w/w | % w/w |
| Water | 90.00 | 90.00 |
| PG | 10.00 | |
| IPA | | 10.00 |
| TOTAL | 100.00 | 100.00 |

### Preparation of Epidermal Sheet

Human epidermal membrane was prepared from skin post cosmetic reduction surgery (abdominoplasty). Full thickness skin was defrosted at ambient temperature until malleable. The subcutaneous fat was removed mechanically by blunt dissection. Upon removal of the fat, skin was immersed in hot deionised water (60±3° C.) for 45 s. The epidermal membrane (comprising the Stratum corneum and epidermis) was removed from the underlying dermis using a gloved finger and the dermis was discarded. The epidermal membrane was then floated (Stratum corneum side up) in deionised water onto filter paper. Excess water was removed from the surface and the tissue was stored until required.

### Effect of a 15 Minute Application of Heat on the Permeation of Three Model Compounds.

Individually calibrated unjacketed small Franz type diffusion cells were employed. Each cell had an average surface area approximately 0.60 cm² and a volume of approximately 2.0 mL Studies were conducted at two temperatures, so the surface temperature of the skin would be maintained at 32° C. and 45° C. for fifteen minutes, then replaced into the 32° C. water bath.

A total of 50 cells were prepared, with four active formulations, n=6 at each condition and one blank cell with no formulation applied. The lower receptor chamber was filled with respective receiver fluid (acyclovir: phosphate buffered saline solution, sildenafil citrate: phosphate buffered saline solution and diclofenac: phosphate buffered saline solution). Each cell was then inverted and observed for leaks. If no leaks were observed the cells were used, if leaks were observed, the cell was remounted.

Cells were placed in the water bath and allowed to equilibrate for 30 min prior to dosing. A 0.5 g dose of each formulation was applied to twelve cells using a pre-calibrated positive displacement pipette. Six cells remained at a constant membrane temperature of 32° C., the other six were heated in a water bath for 15 minutes to obtain a membrane temperature of 45° C. (and then returned to the other water bath to achieve a constant membrane temperature of 32° C. Following application of the donor formulation, each cell was occluded to prevent solvent evaporation. Samples of receiver fluid (200 μL) were removed from the sampling arm at 0, 0.25, 0.5, 1, 2, 4, 6, 24, 27, 30 and 48 h time points (where applicable). After each sample was removed, an equal volume of pre-warmed receiver fluid was replaced. Samples were analysed using HPLC/UPLC as defined above.

### Primary Embodiment—Two Part Semi-Solid Systems

The exothermic supercooled salts (sodium acetate and sodium thiosulphate) were investigated as a method to generate heat to increase permeation. A non-heat generating salt at the same concentration was used as the control in the non-heat generating systems. A secondary permeation method was used to obtain data using the exothermic salts assessed earlier. Individually calibrated unjacketed large Franz type diffusion cells were employed. Each cell had a surface area of approximately 2.0 cm² and a volume of approximately 10 mL. The study was conducted using the diclofenac donor solutions detailed above together with the highest saturation of sodium thiosulphate and sodium acetate solutions.

A total of 50 cells were prepared with four active donor gels, n=3 with both the heat generating and non-heat generating salt and a blank. The integrity of the epidermal membrane was assessed visually prior to dosing the cells. The lower receptor chamber was filled with phosphate buffered saline solution.

Cells were placed dosed with 0.5 mL of the donor gels prepared above, followed by the salt solution (2.5 mL) and a seed crystal to initiate heat generation. The exothermic crystallisation mimicked the effect of the previous permeation, raising the skin surface temperature to ca. 45° C. for approximately 15 minutes. For the non-heated cells a mimetic salt at equivalent molarity was used in place of the exothermic salt. Following application of the donor formulation, each cell was occluded to prevent solvent evaporation and the ingress of water. Samples of receiver fluid (1 mL) were removed from the sampling arm at 0, 0.5, 1, 2, 4, 6, 24, 27 and 30 h. After each sample was removed, an equal volume of pre-warmed receiver fluid was replaced. Samples were analysed using the diclofenac HPLC method detailed above.

### Diclofenac Gel Formulations

Donor gels were prepared by saturating the solvent system with the API for approximately 12 h. The quantity of API required for each system was predicted using the provisional API solubility tests. It was ensured that a fine precipitate of API was observed in each donor gel prior to dosing in the in vitro permeation experiments. Carbopol ultrez was used at 1.0% w/w to ensure a slightly increased viscosity was achieved. This enabled the gel to stay in contact with the epidermis throughout the permeation without being displaced by the supercooled salts.

| | System 1 | System 2 | System 3 | System 4 |
|---|---|---|---|---|
| Excipient | % w/w | % w/w | % w/w | % w/w |
| Water | 24.50 | 19.67 | 19.67 | 19.75 |
| PEG-400 | 74.50 | 69.67 | 69.67 | 69.75 |
| EtOH | 0.00 | 9.66 | 0.00 | 4.75 |
| Arlasolve DMI | 0.00 | 0.00 | 9.66 | 4.75 |
| Carbopol Ultrez | 1.00 | 1.00 | 1.00 | 1.00 |

### Two Part Patch System Using a Supercooled Solution

Individually calibrated, unjacketed small Franz type diffusion cells were employed without donor chambers. Epidermal membrane was fixed to each cell using cyanoacrylate adhesive to allow for a patch to be applied directly over the gel formulation applied to the epidermis. Each cell had a surface area of ca. 0.60 cm² and a volume of 2.0 mL.

The lower receptor chamber was filled with phosphate buffered saline solution, Once checked, a rubber O-ring was fixed to the epidermis using cyanoacrylate adhesive, creating an area for gel application that would not be lost over the experimental period.

Each cell was dosed with the donor gel (0.25 mL) and covered with Parafilm® to contain the gel. The heat patch was positioned above the gel and initiated for heated cells. Samples of receiver fluid (1 mL) were removed from the sampling arm at 0, 0.5, 1, 2, 4, 6, 24 and 30 h time points. After each sample was removed, an equal volume of pre-warmed receiver fluid was replaced. Samples were analysed using HPLC using the diclofenac method detailed above.

### Example 4

### Results

### Assessment of Thermogenic Materials

Three different methods to generate heat were investigated and characterised, in order to establish appropriate heat sources for the present invention. The aim was to find heat sources that could provide an initial burst of heat at ca. 42° C. for ca. 15 minutes.

### Heat of Hydration

The heating profiles of the two different anhydrous salts, MgSO₄ or CaCl₂, were recorded over 10 min after the hydration reaction was initiated by the addition of water. Three indices were taken from the temperature profile: the peak of the maximum temperature change (Tₘₐₓ), time to reach the maximum temperature (tₘₐₓ) and duration (DUR₃₂), defined as the time taken to lose 3° C. from the maximum temperature (Table 13).

TABLE 13
Tₘₐₓ, tₘₐₓ and DUR from hydration of MgSO₄ or CaCl₂

| | MgSO₄ (0.5 g) | | | CaCl₂ (0.5 g) | | |
|---|---|---|---|---|---|---|
| | Tₘₐₓ (° C.) | tₘₐₓ (s) | DUR₃₂ (s) | Tₘₐₓ (° C.) | tₘₐₓ (s) | DUR₃₂ (s) |
| 1 mL | 42.6 ± 1.4 | 45 ± 0 | 57 ± 7 | 39.9 ± 0.9 | 60 ± 11 | 93 ± 16 |
| 2 mL | 39.8 ± 1.4 | 57 ± 7 | 123 ± 34 | 34.2 ± 0.8 | 115 ± 12 | 384 ± 54 |
| | MgSO₄ (1 g) | | | CaCl₂ (1 g) | | |
| | Tₘₐₓ (° C.) | tₘₐₓ (s) | DUR₃₂ (s) | Tₘₐₓ (° C.) | tₘₐₓ (s) | DUR₃₂ (s) |
| 2 mL | 50.7 ± 1.5 | 48 ± 13 | 60 ± 0 | 41.0 ± 0.8 | 87 ± 20 | 210 ± 102 |
| 5 mL | 43.0 ± 0.5 | 39 ± 8 | 156 ± 48 | 33.3 ± 0.5 | 132 ± 27 | 465 ± 0 |
| | MgSO₄ (2 g) | | | CaCl₂ (2 g) | | |
| | Tₘₐₓ (° C.) | tₘₐₓ (s) | DUR₃₂ (s) | Tₘₐₓ (° C.) | tₘₐₓ (s) | DUR₃₂ (s) |
| 2 mL | 64.8 ± 2.1 | 27 ± 13 | 60 ± 24 | 53.0 ± 1.1 | 57 ± 20 | 159 ± 50 |
| 5 mL | 56.6 ± 2.9 | 63 ± 13 | 90 ± 30 | 41.6 ± 1.7 | 99 ± 85 | 315 ± 19 |

Increasing the amount of both salts from 0.5 to 2.0 g, while keeping the volume of water used for the hydration reaction at 2 mL, increased Tₘₐₓ from 39.8° C. to 64.2° C. (for MgSO₄) and from 34.3° C. to 52.7° C. (for CaCl₂) (FIGS. 16 and 17). However, increasing the volume of water used, without changing the amount of the salt, Tₘₐₓ decreased (FIGS. 18 and 19). This was thought to be attributed to the volume effect of water in conjunction with the heat capacity of water, where a greater volume of water would require more energy to heat to the same temperature as the lesser volume of water. The Tₘₐₓ for each salt was achieved within 1 minute. The rapid rise to maximum temperature and max temperature produced would suggest that the heat of hydration is a suitable means of generating heat at a profile required to increase skin lipid fluidity within the SC.

### Enthalpy of Polymerisation

The temperature profile of the polymerisation reaction, initiated in the presence of bisphenol-A (BPA) with epichlorohydrin (ECH), was recorded over 60 min. Tₘₐₓ and tₘₐₓ were respectively 40.2±1.4° C. and 10±0 min (FIG. 20). Over the 60 minute period the temperature was observed to remain above 32° C. Although not as rapid as the increase in temperature observed with heat of hydration, the temperature generated from the polymeric system was at a suitable level for the present invention.

### Supercooled Salt Solutions

### Temperature Profiling of Exothermic Salts

The highest temperatures achieved were observed by the salt solutions with the highest saturation level. A volume effect was also observed, where increasing the volume from 1 mL to 2.5 mL increased the maximum temperature (Tₘₐₓ) by ca. 10° C. (Table 14). The maximum temperature achieved was 42.6° C. by the solution at the highest saturation level (10.0 g sodium acetate to 8.0 g water) of sodium acetate with a volume of 2.5 mL. The time taken to reach the maximum temperature (tₘₐₓ) was 118 seconds and the duration the formulation remained above 32° C. was in excess of 10 min. The second highest saturation level of sodium acetate (10.0 g sodium acetate to 9.0 g water) at the same volume had a comparable Tₘₐₓ (41.1° C.) that was reached in 116 seconds. The duration the solution remained above 32° C. (DUR₃₂) was observed to be greater than 9 min. As the volume level decreased the Tₘₐₓ and duration were also observed to decrease

Based on the data and the maximum volume that could be applied to a Franz diffusion cell the solution with a ratio of 10 g salt to 8 g water (supercooled solution at the highest saturation) solution at a volume of 2.5 mL was selected.

TABLE 14
Temperature assessment of sodium acetate at
different molarities and volumes, n = 1.

| salt (g):water | Volume | Tₘₐₓ | Tₘₐₓ | DUR₃₂ | AUC₃₂ |
|---|---|---|---|---|---|
| (g) ratio | (mL) | (° C.) | (secs) | (minutes) | (sec · degree) |
| 10:8 | 1 | 38.7 | 90 | 4.72 | 1193 |
| | 2.5 ml | 42.6 | 118 | 10.38 | 3934 |
| 10:9 | 1 | 33.9 | 83 | 2.15 | 155 |
| | 2.5 ml | 41.1 | 116 | 9.50 | 2925 |
| 10:10 | 1 | 37.7 | 44 | 3.87 | 777 |
| | 2.5 ml | 44.3 | 46 | 9.71 | 3584 |
| 10:11 | 1 | 32.4 | 82 | 1.56 | 69 |
| | 2.5 ml | 41.1 | 51 | 9.28 | 2657 |
| 10:12 | 1 | 32.5 | 59 | 1.15 | 53 |
| | 2.5 ml | 35.9 | 55 | 5.32 | 687 |
| 10:13 | 1 | 31.6 | 39 | 0.59 | 13 |
| | 2.5 ml | 35.0 | 71 | 5.41 | 701 |
| 10:14 | 1 | 28.2 | 41 | 0 | 0 |
| | 2.5 ml | 31.0 | 52 | 0.67 | 19 |
| 10:15 | 1 | 26.9 | 53 | 0 | 0 |
| | 2.5 ml | 29.2 | 53 | 0 | 0 |
| 10:16 | 1 | 24.5 | 149 | 0 | 0 |
| | 2.5 ml | 27.4 | 56 | 0 | 0 |

A volume effect similar to that observed with sodium acetate was still observed with sodium thiosulphate between the 1 mL and 2.5 mL solutions. The highest temperature was achieved by the solution containing the ratio of 10.0 g salt to 5.0 g water at a volume of 2.5 mL. The solution which reached a Tₘₐₓ of 45.8° C. and managed reach this temperature within 145 seconds before cooling. As well as maintaining a temperature above 32° C. for over 18 minutes (DUR₃₂—18.65 minutes).

These results demonstrated that a 2.5 mL volume of sodium thiosulphate is sufficient to obtain the desired heating effect. As such the system was selected for investigation in the permeation studies.

TABLE 15
Temperature assessment of Sodium thiosulphate
using a range of molarities and volumes, n = 1.

| salt (g):water | Volume | Tₘₐₓ | Tₘₐₓ | DUR₃₂ | AUC₃₂ |
|---|---|---|---|---|---|
| (g) Ratio | (mL) | (° C.) | (secs) | (minutes) | (sec · degree) |
| 10:5 | 1 | 36.9 | 178 | 6.48 | 1303 |
| | 2.5 | 45.8 | 145 | 18.65 | 11724 |
| 10:6 | 1 | 37.7 | 153 | 7.07 | 1703 |
| | 2.5 | 41.8 | 185 | 21.15 | 9664 |
| 10:7 | 1 | 35.3 | 124 | 5.6 | 797 |
| | 2.5 | 43.1 | 91 | 14.43 | 5845 |
| 10:8 | 1 | 36.3 | 56 | 5.13 | 802 |
| | 2.5 | 36.8 | 82 | 13.41 | 1738 |
| 10:9 | 1 | 27.4 | 122 | 0 | 0 |
| | 2.5 | 32.9 | 74 | 2.82 | 143 |
| 10:10 | 1 | 26.1 | 152 | 0 | 0 |
| | 2.5 | 27.2 | 176 | 0 | 0 |
| 10:11 | 1 | 23.6 | 126 | 0 | 0 |
| | 2.5 | 25.2 | 177 | 0 | 0 |

### Analytical Method Development

Typical chromatograms for sildenafil citrate, diclofenac and acyclovir were obtained (not shown). Results were obtained by integrating the area under the principal eluted peak, which was found to have a retention time of approximately 5, 7 and 5/10 (standards and samples) min for sildenafil, diclofenac and acyclovir, respectively. Each calibration curve had a linearity of r²≦0.99. The limit of quantification (LOQ) were; 0.10 μg/ml for sildenafil citrate (between 0.05 and 2 μg/ml), 0.23 μg/ml for diclofenac (between 0.5 and 10 μg/ml) and 0.17 μg/ml for acyclovir (between 0.05 and 10 μg/ml). It was concluded that the HPLC methodology developed was ‘fit for purpose’ for all three API's.

### API Solubility

The saturated solubility of each test item is summarised in Table 16. Based on the data previously obtained (from literature values of solubility and previous studies) the composition of the solvents within the donor formulations for each API was developed. Solvents within each formulation were selected on the basis of high API solubility and visual compatibility between excipients.

TABLE 16
Visually determined saturated solubility
of each test item in various solvents.

| | Sildenafil citrate | Acyclovir | Diclofenac free acid |
|---|---|---|---|
| Solvent | (mg/g) | (mg/g) | (mg/g) |
| MeOH | 6.60-10.02 | 0.29-0.62 | 34.43-68.06 |
| EtOH | 0.54-1.06 | <0.35 | 31.60-62.79 |
| Phenoxyethanol | 6.56-9.88 | 0.54-1.08 | 34.32-81.12 |
| PEG-400 | 3.85-6.32 | 0.799-1.07 | 28.83-51.73 |
| PG | 1.90-2.86 | 1.66-2.39 | 10.15-20.50 |
| Diethyl Ether | <0.90 | <0.54 | 21.28-49.89 |
| Isoamyl Alcohol | <0.45 | <0.31 | 34.25-53.55 |
| Arlasolve DMI | <0.88 | <0.29 | 57.90-110.45 |
| IPA | <1.27 | <0.34 | 29.97-58.09 |
| Transcutol P | 2.14-2.85 | <0.30 | 108.73-162.17 |

Following visual solubility assessment of the API in individual solvents, donor solutions were derived to ensure thermodynamic activity was not a variable a fine suspension of the API was included in all donor systems used in the permeation studies.

### Example 5

### API In Vitro Permeation Studies

### Effect of a 15 Minute Application of Heat on the Permeation of Three Model Compounds

The permeation of each donor formulation was assessed at a constant membrane temperature of 32° C. and with 15 minutes at an elevated membrane temperature of 45° C. before being equilibrated at a membrane temperature of 32° C. Statistical analysis between the data has not been performed due to the small n numbers used in the experiments as such all data is represented by a mean value.

### In Vitro Permeation of Sildenafil Citrate Through Epidermal Membrane

Despite only an initial application of heat the greatest differences between the formulations were observed at 4 h (FIG. 21). The formulation that resulted in the highest delivery of sildenafil citrate through epidermal membrane was the formulation that contained the chemical enhancers Transcutol P and methanol in combination and the membrane temperature was elevated for the initial 15 minutes of the experiment (Formulation: Water:TP:MeOH (elevated)). At t=4 h the cumulative amount of sildenafil permeated was 5.19 μg/cm² which was observed to be nearly 4 times higher (enhancement 3.87) than the permeation of sildenafil from the same formulation without an initial elevated membrane temperature which was observed to be 1.59 μg/cm² (FIG. 22). Furthermore, the formulation that contained no enhancers, but was heated, was observed to have markedly higher sildenafil permeation than the formulation the contained no penetration enhancers and was not heated (1.18 μg/cm²) at the same time point. The enhancement in permeation of sildenafil citrate at t=4 h in comparison to the non-heated formulation with no penetration enhancers (Formulation: water) was observed to be 1.51-fold for the heated formulation (elevated), 1.14-fold for the same formulation with enhancers and 4.40-fold for the formulation of the present invention, with heat and enhancers.

### In Vitro Permeation of Acyclovir Through Epidermal Membrane

The effect of heat was observed to increase permeation of acyclovir, in comparison to the same formulation without heat, as illustrated in the t=2 h data (FIG. 23). For example the permeation of acyclovir from the lead formulation (H₂O:IPA (elevated)) was 10.59 μg/cm² which when compared to the same formulation not heated showed an 1.38-fold enhancement. A similar trend was observed between H₂O:PG non-heated and heated, (5.62 μg/cm² and 7.85 μg/cm²) with a 1.40-fold enhancement. The results demonstrate a beneficial effect of heat in combination with enhancers for acyclovir.

### In Vitro Permeation of Diclofenac Free Acid Through Epidermal Membrane

Diclofenac permeation was observed to be notably higher for the solutions containing both enhancers and that had an initial 15 minute elevated temperature. The formulations followed the expected trend of, heat and enhancer having a greater permeation than heat alone which in turn had a greater permeation than enhancer alone generally with a concomitant reduction in time of onset (FIG. 24). For example, diclofenac was quantified in the receiver fluid at 15 min H₂O/PEG:EtOH (elevated) but 30 min for H₂O/PEG:EtOH alone. Whilst for H₂O/PEG, diclofenac was first quantified at 2 h at elevated temperature compared to 6 h with no heat.

At 8 h, notably higher diclofenac permeation was observed from two formulations with heat through the human epidermis in comparison to the formulation with no heat and no enhancers (H₂O/PEG) where the mean cumulative permeation of diclofenac was observed to be 0.17 μg/cm² (FIG. 25). The mean diclofenac permeation increased by 1.6-fold with the application of heat (H₂O/PEG (elevated)) to 0.27 μg/cm², using chemical enhancers alone (H₂O/PEG:EtOH) increased the permeation by 6.4-fold (1.08 μg/cm²). However, when chemical enhancers and heat were used together (H₂O/PEG:EtOH (elevated)) the diclofenac permeation was observed to increase by 10.2-fold (1.73 μg/cm²) in comparison to the formulation with no heat and no enhancers (H₂O/PEG).

### i) Two Part Semi-Solid Systems Using Exothermic Supercooled Solutions

Diclofenac Permeation from Two Part Semi-Solid Systems Using Supercooled Sodium Thiosulphate

In order to demonstrate that this embodiment would work with the methodology of the present invention, the salt sodium thiosulphate was used.

The effect of heat was observed to increase permeation of diclofenac, in comparison to the same formulation without heat, as illustrated in the cumulative permeation of diclofenac at t=6 h (FIG. 26). For example the cumulative permeation of diclofenac from the formulation H₂O:PEG:EtOH heated using sodium thiosulphate was 0.18 μg/cm² after 6 h in comparison to the same formulation with no pre-heating, where no diclofenac permeation was observed. The permeation of diclofenac from the lead formulation H₂O:PEG:EtOH:Arlasolve DMI heated using sodium thiosulphate was 0.49 μg/cm² which compared to the same formulation not heated showed a 2.5-fold enhancement.

Diclofenac Permeation from Two Part Semi-Solid Systems Using Supercooled Sodium Acetate

The permeation of diclofenac from formulations using sodium acetate to generate heat was observed to be markedly higher than the same formulation without heat (FIG. 27). Overall the permeation of diclofenac from the formulation H₂O/PEG:arlasolve DMI at an elevated temperature with sodium acetate was the highest, where 7.01 μg/cm² had permeated (an enhancement of 23.56-fold compared to the heated formulation with no enhancers as no permeation was observed from the non-heated formulation with no enhancers). In comparison, after 6 h only 0.71 μg/cm² of diclofenac had permeated from the same formulation without heat (9.87-fold enhancement). The formulation containing ethanol and arlasolve DMI was also observed to have notably higher diclofenac permeation in comparison to the respective formulation without heat, a 5.27-fold increase.

### ii) Two Part Patch Systems Using Supercooled Solutions

A second embodiment of a supercooled solution of sodium acetate contained within a patch was investigated as a method to generate heat to enhance the permeation of diclofenac from two semi-solid gel preparations. The patch reached a maximum temperature of ca. 42° C. and remained above 32° C. for ca. 30 minutes. The patch was applied above the gel, and initiated to produce heat. The permeation of diclofenac from each formulation with and without heat was relatively linear (r²>0.91) for the first 24 h of the experimental duration, at the 30 h time point an increase in diclofenac permeation from each formulation was observed (FIG. 28). The onset of diclofenac permeation was observed at 6 h from the formulation with no heat and no chemical enhancers (H₂O/PEG-400), at 2 h from the formulation containing chemical enhancers (H₂O/PEG-400:EtOH) and at 1 h from the formulation containing heat and chemical enhancers (H₂O/PEG-400:EtOH elevated). The outcomes confirm that the application of heat to a formulation containing chemical enhancers increases drug permeation and markedly reduces the onset time of permeation.

The most notable difference was observed at 30 h (FIG. 29) where the mean cumulative permeation of diclofenac from the heating formulation containing chemical enhancers (H₂O/PEG-400:EtOH elevated) was 5.37 μg/cm² a 3.09-fold enhancement in comparison to no heat and no chemical enhancers (H₂O/PEG-400) where the permeation of diclofenac from the formulation was 1.74 μg/cm². Furthermore enhancements in diclofenac permeation of 1.33-fold and 1.49-fold were observed for the formulation containing chemical enhancers alone (H₂O/PEG-400:EtOH, 2.31 μg/cm²) and the formulation that contained no chemical enhancers but was heated (H₂O/PEG-400 elevated, 2.59 μg/cm²), in comparison to the formulation that had no enhancers and was not heated.

### Conclusions

A variety of methods to generate heat for the purpose of elevating skin temperature for use in the present invention was investigated. The maximum temperatures attained by the methods of heat generation investigated were sufficiently high and, without being bound by theory, it is believed that the temperatures reached were able to induce lipid phase transitions within the skin (ca. 42° C.).

The formulation that resulted in the highest delivery of sildenafil citrate through epidermal membrane contained enhancers Transcutol P and methanol, and was elevated in temperature for the initial 15 minutes of the experiment. At t=4 h the cumulative amount of sildenafil permeated was observed to be almost 4-fold (3.87 enhancement) higher than the permeation of sildenafil from the same formulation without an initial elevated membrane temperature.

The effect of heat was observed to increase permeation of acyclovir, in comparison to the same formulation without heat, as illustrated in the t=2 h data. For example the permeation of acyclovir from the lead formulation (H₂O:IPA) was 10.59 μg/cm² which when compared to the same formulation not heated showed an 1.38-fold enhancement. A similar enhancement between H₂O:PG non-heated and heated of 1.40-fold was observed.

Diclofenac permeation was observed to be notably higher for the solutions containing both enhancers and that had an initial 15 minute elevated temperature. The formulations followed the overall trend of heat and enhancer in combination having a greater permeation than heat alone which, in turn, had a greater permeation than enhancer alone, which had greater permeation than no heat and no enhancer. At 8 h, increased diclofenac permeation was observed by 1.6-fold with the application of heat, 6.4-fold using chemical enhancers alone and 10.2-fold when chemical enhancers and heat were used together in comparison to the formulation with no heat and no enhancers. Due to the clear trends observed with the application of heat and use of chemical enhancers within the diclofenac formulations, diclofenac was selected for use within two embodiments using supercooled solutions to generate heat.

The first embodiment tested was a two part semi-solid formulation, with a supercooled solution of sodium acetate applied above a gel containing diclofenac. The permeation of diclofenac from formulations using sodium acetate to generate heat was observed to be markedly higher than the same formulation without heat. Overall the permeation of diclofenac from the formulation H₂O/PEG:arlasolve DMI at an elevated temperature (with sodium acetate) was the highest with an enhancement of 23.56-fold compared to the heated formulation with no enhancers. In comparison, only a 9.87-fold enhancement was observed from the same formulation without heat.

A second embodiment involved a supercooled solution of sodium acetate contained within a patch as a method to generate heat to enhance the permeation of diclofenac from semi-solid gel preparations. The patch reached a maximum temperature of ca. 42° C. and remained above 32° C. for ca. 30 minutes. The most notable difference was observed at 30 h where the mean cumulative permeation of diclofenac from the heating formulation containing chemical enhancers was 3.09-fold higher than the formulation that had no heat and no chemical enhancers.

Thus, an initial burst of heat, such as can be generated by the heat of hydration, enthalpy of polymerisation and initiation of supercooled salts, coupled with one or more permeation enhancers, has been shown to be sufficient to disrupt the barrier integrity of the SC and to increase drug permeation.

### REFERENCES

Akomeah, F., Nazir, T., Martin, G. P., & Brown, M. B. 2004. Effect of heat on the percutaneous absorption and skin retention of three model penetrants. European Journal of Pharmaceutical Sciences, 21 (2-3), 337-345.

Ashburn, M. A., Ogden, L. L., Zhang, J., Love, G., & Basta, S. V. 2003. The pharmacokinetics of transdermal fentanyl delivered with and without controlled heat. The Journal of Pain, 4 (6), 291-297.

Blank, I. H., Scheuplein, R. J., & Macfarlane, D. J. 1967. Mechanism of Purcutaneous Absorption III. The effect of temperature on the transport of non-electrolytes across the skin. The Journal of Investigative Dermatology, 49 (6), 582-589.

Bos, J. D. & Meinardi, M. M. H. M. 2000. The 500 Dalton rule for the skin penetration of chemical compounds and drugs”, Experimental Dermatology, 9 (3), 165-169.

Chernomordik, L. V., Papahadjopoulos, D., & Tsong, T. Y. 1991. Increased binding of liposomes to cells by electric treatment. Biochimica et Biophysica Acta (BBA)—Biomembranes, 1070 (1), 193-197.

Choi, M. J. & Maibach, H. I. 2005. Liposomes and Niosomes as Topical Drug Delivery Systems. Skin Pharmacology and Physiology, 18, 209-219.

Finnin, B. C. & Morgan, T. M. 1999. Transdermal penetration enhancers: Applications, limitations, and potential, Journal of Pharmaceutical Sciences, 88 (10), 955-958.

Gay, C. L., Guy, R. H., Golden, G. M., Mak, V. H. W., Francoeur, M. L., 1994. Characterization of low temperature (<65° C.) lipid transitions in human stratum corneum. J. Invest. Dermatol., 103 (2), 233-239.

Hadgraft, J. 2001. Skin, the final frontier, International Journal of Pharmaceutics, 224 (1-2), 1-18.

Harrison, J. E., Groundwater, P. W., Brain, K. R., Hadgraft, J., 1996. Azone® induced fluidity in human stratum corneum. A fourier transform infrared spectroscopy investigation using the perdeuterated analogue. J. Control Release, 41, 283-290.

Hull, W. 2002. Heat-enhanced transdermal drug delivery: a survey paper, Journal of Applied Research, 2 (1), 1-9.

Iervolino, M., Cappello, B., Raghavan, S. L., & Hadgraft, J. 2001. Penetration enhancement of ibuprofen from supersaturated solutions through human skin, International Journal of Pharmaceutics, 212 (1), 131-141.

Iervolino, M., Raghavan, S. L., & Hadgraft, J. 2000. Membrane penetration enhancement of ibuprofen using supersaturation, International Journal of Pharmaceutics, 198 (2), 229-238.

Klemsdal, T. O., Gjesdal, K., & Bredesen, J. E. 1992. Heating and Cooling of the Nitroglycerin Patch Application Area Modify the Plasma-Level of Nitroglycerin, European Journal of Clinical Pharmacology, 43 (6), 625-628.

Kligman, A. M., Christophers, E., 1963. Preparation of isolated sheets of human stratum corneum. Arch. Dermatol., 88, 702-705.

Moser, K., Kriwet, K., Naik, A., Kalia, Y. N., & Guy, R. H. 2001. Passive skin penetration enhancement and its quantification in vitro, European Journal of Pharmaceutics and Biopharmaceutics, 52 (2), 103-112.

Naik, A., Kalia, Y. N., & Guy, R. H. 2000. Transdermal drug delivery: overcoming the skin's barrier function, Pharmaceutical Science & Technology Today, 3 (9), 318-326.

Ogiso, T., Hirota, T., Iwaki, M., Hino, T., & Tanino, T. 1998. Effect of temperature on percutaneous absorption of terodiline, and relationship between penetration and fluidity of the stratum corneum lipids, International Journal of Pharmaceutics, 176 (1), 63-72.

Prausnitz, M. R. 2004, Microneedles for transdermal drug delivery, Advanced Drug Delivery Reviews, 56 (5), 581-587.

Prausnitz, M. R., Mitragotri, S., & Langer, R. 2004. Current Status and Future Potential of Transdermal Drug Delivery, Nature Reviews Drug Discovery, 3 (2), 115-124.

Scheuplein, R. J. & Blank, I. H. 1971. Permeability of the Skin, Physiological Reviews, 51 (4), 702-747.

Scheuplein, R. J. 1965. Mechanism of Percutaneous Adsorption.I. Routes of Penetration and Influence of Solubility, Journal of Investigative Dermatology, 45 (5), 334-.

Shomaker, T. S., Zhang, J., & Ashburn, M. A. 2001. A Pilot Study Assessing the Impact of Heat on the Transdermal Delivery of Testosterone, Journal of Clinical Pharmacology, 41, 677-682.

Shomaker, T. S., Zhang, J., & Ashburn, M. A. 2000. Assessing the impact of Heat on the Systemic Delivery of Fentanyl Through the Transdermal Delivery System, Pain Medicine, 1 (3), 225-230.

Silva, C. I., Nunes, S. C. C., Eusebio, M. E. S., Pais, A. A. C. C., Sousa, J. J. S., 2006. Thermal behavior of human stratum corneum. Skin Pharmacol. Physiol., 19, 132-139.

Stanley, T., Hull, W., Rigby, L., & Zhang, J. 2002. Transdermal drug patch with attached pocket for controlled heating device, U.S. Pat. No. 6,488,959B2 (patent).

Teo, A. L., Shearwood, C., Ng, K. C., Lu, J., & Moochhala, S. 2006. Transdermal microneedles for drug delivery applications”, Materials Science and Engineering: B, 132 (1-2) 151-154.

Tiwary, A. K., Sapra, B., & Jain, S. 2007. Innovations in Transdermal Drug Delivery: Formulations and Techniques, Recent Patents on Drug Delivery & Formulation, 1, 23-36.

Wang, Y., Thakur, R., Fan, Q., & Michniak, B. 2005. Transdermal iontophoresis: combination strategies to improve transdermal iontophoretic drug delivery, European Journal of Pharmaceutics and Biopharmaceutics, 60 (2), 179-191.

Wiechers, J. W., Drenth, B. F. H., Jonkman, J. H. G., & de Zeeuw, R. A. 1988. Percutaneous absorption, metabolism, and elimination of the penetration enhancer Azone in humans after prolonged application under occlusion, International Journal of Pharmaceutics, 47 (1-3), 43-49.

Williams, A. C. & Barry, B. W., 2004. Penetration enhancers, Advanced Drug Delivery Reviews, 56 (5), 603-618.

Zars Pharma Ltd. Zars Products. http://www.zars.com/. 2006. 17-10-2006.

1-27. (canceled)

28. A thermogenic formulation for use in the transdermal administration of a drug, wherein said drug is present in said formulation or in a second formulation, one or more penetration enhancers being present in one or both formulations, and wherein at least one penetration enhancer is a C₁₋₁₂ compound comprising at least one -Alk-O— group, in which Alk is a C₁₋₆ alkylene and, when there is only one -Alk-O— group, then Alk may also represent a C₁₋₁₀ alkyl group.

29. The formulation of claim 28, wherein said thermogenic formulation comprises a supercooled salt solution.

30. The formulation of claim 28, wherein said thermogenic formulation is formed in situ or immediately before use by combining a solvent, preferably water, with at least one anhydrous salt capable of yielding heat of hydration, the salts preferably being selected from the anhydrous forms of the salts calcium chloride (CaCl₂) and magnesium sulphate (MgSO₄).

31. The formulation according of claim 28, wherein said thermogenic formulation comprises monomers capable of yielding heat of polymerisation on the addition of a suitable catalyst, the monomers preferably being bisphenol-A (BPA) and epichlorohydrin (ECH).

32. The formulation of claim 28, wherein the thermogenic formulation is capable of reaching temperatures of at least 40° C., and preferably no more than 50° C.

33. The formulation of claim 32, wherein the thermogenic formulation is adapted to reach a temperature of between 42° C. and 45° C. inclusive.

34. The formulation of claim 32 or 33, wherein the thermogenic formulation is adapted to remain above 32° C. for at least 5, preferably at least 10 minutes, and preferably for 15 minutes or more.

35. The formulation of claim 28, wherein -Alk-O— is an H-Alk-O— group, such as H-Alk-OH.

36. The formulation of claim 28, wherein -Alk-O— is -Alk-O-Alk- or a corresponding oligomer, optionally with one or more different comonomeric units.

37. The formulation of claim 28, wherein the penetration enhancer is diethylene glycol monoethyl ether or dimethyl isosorbide.

38. The formulation of claim 28, wherein the penetration enhancer is a C₁₋₁₀ alkanol.

39. The formulation of claim 28, wherein the penetration enhancer is a C₁₋₆ alkanol.

40. The formulation of claim 28, wherein the thermogenic formulation further comprises the penetration enhancer.

41. The formulation of claim 28, wherein the penetration enhancer is a C₁₋₃ alkanol, especially methanol, ethanol, or IPA, most preferably ethanol.

42. The formulation of claim 28, wherein the thermogenic formulation is a solution of a supercooled salt, and the salt is the hydrate of sodium thiosulphate or preferably of sodium acetate.

43. The formulation of claim 28, wherein the thermogenic formulation is a solution of a supercooled salt, and wherein a crystallisation nucleant is present in the drug formulation.

44. The formulation of claim 28, wherein the thermogenic formulation is a solution of a supercooled salt, and further comprises benzyl alcohol.

45. The formulation of claim 28, in the form of a transdermal patch comprising an adhesive for application to the skin and a reservoir containing a solution of a supercooled salt, the drug and enhancer being comprised in the adhesive, and an impermeable membrane separating the salt solution and the adhesive.

46. The formulation of claim 28, wherein the thermogenic formulation is a solution of a supercooled salt, and wherein the formulation comprising the salt is provided as a liquid or gel or gum.

47. The formulation of claim 46, wherein the formulation comprising the salt is supplied in a squeezable tube, liquid dispenser or preferably an aerosol dispenser.

48. A formulation comprising a supercooled solution of a salt, for use in the transdermal administration of a drug, said salt being capable of releasing heat of crystallisation and wherein said drug is administered as part of said formulation or as a second formulation, a penetration enhancer being present in one or both formulations, wherein said penetration enhancer is a C₁₋₁₂ compound comprising at least one -Alk-O— group, in which Alk is a C₁₋₆ alkylene and, when there is only one -Alk-O— group, then Alk may also represent a C₁₋₁₀ alkyl group.

49. The formulation of claim 48, wherein -Alk-O— is an H-Alk-O— group, such as H-Alk-OH.

50. The formulation of claim 48, wherein -Alk-O— is -Alk-O-Alk- or a corresponding oligomer, optionally with one or more different comonomeric units.

51. The formulation of claim 48, wherein the penetration enhancer is diethylene glycol monoethyl ether or dimethyl isosorbide.

52. The formulation of claim 48, wherein the penetration enhancer is a C₁₋₁₀ alkanol.

53. The formulation of claim 48, wherein the penetration enhancer is a C₁₋₆ alkanol.

54. The formulation of claim 48, wherein the thermogenic formulation further comprises the penetration enhancer.

55. The formulation of claim 48, wherein the penetration enhancer is a C₁₋₃ alkanol, especially methanol, ethanol, or IPA, most preferably ethanol.

56. The formulation of claim 48, wherein the thermogenic formulation is a solution of a supercooled salt, and the salt is the hydrate of sodium thiosulphate or preferably of sodium acetate.

57. The formulation of claim 48, wherein the thermogenic formulation is a solution of a supercooled salt, and wherein a crystallisation nucleant is present in the drug formulation.

58. The formulation of claim 48, wherein the thermogenic formulation is a solution of a supercooled salt, and further comprises benzyl alcohol.

59. The formulation of claim 48, in the form of a transdermal patch comprising an adhesive for application to the skin and a reservoir containing a solution of a supercooled salt, the drug and enhancer being comprised in the adhesive, and an impermeable membrane separating the salt solution and the adhesive.

60. The formulation of claim 48, wherein the thermogenic formulation is a solution of a supercooled salt, and wherein the formulation comprising the salt is provided as a liquid or gel or gum.

61. The formulation of claim 60, wherein the formulation comprising the salt is supplied in a squeezable tube, liquid dispenser or preferably an aerosol dispenser.

62. A medicament for the transdermal administration of a drug, said medicament comprising separately disposed formulations, a first formulation being a thermogenic formulation, and preferably comprising a supercooled solution of a salt, and a second formulation comprising drug to be topically administered, wherein one or both of said formulations comprises a penetration enhancer as defined in any preceding claim, and wherein said salt, when present, is capable of releasing heat of crystallisation.

63. The medicament of claim 62, wherein said first formulation comprises said penetration enhancer.

64. The medicament of claim 62, wherein the penetration enhancer is a C₁₋₃ alkanol, especially methanol, ethanol, or IPA, most preferably ethanol.

65. The medicament of claim 62, wherein the thermogenic formulation is a solution of a supercooled salt, and the salt is the hydrate of sodium thiosulphate or preferably of sodium acetate.

66. The medicament of claim 62, wherein the thermogenic formulation is a solution of a supercooled salt, and wherein a crystallisation nucleant is present in the drug formulation.

67. The medicament of claim 62, wherein the thermogenic formulation is a solution of a supercooled salt, and further comprises benzyl alcohol.

68. The medicament of claim 62, in the form of a transdermal patch comprising an adhesive for application to the skin and a reservoir containing a solution of a supercooled salt, the drug and enhancer being comprised in the adhesive, and an impermeable membrane separating the salt solution and the adhesive.

69. The medicament of claim 62, wherein the thermogenic formulation is a solution of a supercooled salt, and wherein the formulation comprising the salt is provided as a liquid or gel or gum.

70. The medicament of claim 69, wherein the formulation comprising the salt is supplied in a squeezable tube, liquid dispenser or preferably an aerosol dispenser.

71. A kit comprising the medicament of claim 62 or 63, preferably further comprising a supercooled salt solution and means for initiating crystallisation of said salt.

72. The kit of claim 71, wherein the penetration enhancer is a C₁₋₃ alkanol, especially methanol, ethanol, or IPA, most preferably ethanol.

73. The kit of claim 71, wherein the thermogenic formulation is a solution of a supercooled salt, and the salt is the hydrate of sodium thiosulphate or preferably of sodium acetate.

74. The kit of claim 71, wherein the thermogenic formulation is a solution of a supercooled salt, and wherein a crystallisation nucleant is present in the drug formulation.

75. The kit of claim 71, wherein the thermogenic formulation is a solution of a supercooled salt, and further comprises benzyl alcohol.

76. The kit of claim 71, in the form of a transdermal patch comprising an adhesive for application to the skin and a reservoir containing a solution of a supercooled salt, the drug and enhancer being comprised in the adhesive, and an impermeable membrane separating the salt solution and the adhesive.

77. The kit of claim 71, wherein the thermogenic formulation is a solution of a supercooled salt, and wherein the formulation comprising the salt is provided as a liquid or gel or gum.

78. The kit, of claim 77, wherein the formulation comprising the salt is supplied in a squeezable tube, liquid dispenser or preferably an aerosol dispenser.

79. A physiologically acceptable formulation comprising a C₁₋₆ alkanol and a supercooled solution of a salt, as defined in claim 28.

80. A physiologically acceptable formulation comprising a C₁₋₆ alkanol and a supercooled solution of a salt, as defined in claim 48.

81. A physiologically acceptable formulation comprising a C₁₋₆ alkanol and a supercooled solution of a salt, as defined in claim 64.

82. A physiologically acceptable formulation comprising a C₁₋₆ alkanol and a supercooled solution of a salt, as defined in claim 71.

83. A method for the transdermal administration of a drug, comprising topical administration of a first drug formulation, and subsequent administration of a second thermogenic formulation, preferably comprising a supercooled solution of a salt capable of releasing heat of crystallisation and, thereafter, crystallising in situ, directly onto said first formulation, one or both of said formulations comprising a C₁₋₆ alkanol.

84. A method for enhancing transdermal administration of a drug, comprising localised heating of an area of skin where it is desired to apply drug, heating said area to between 40° C. and 50° C. inclusive, preferably 42° C. to 45° C. inclusive, for a period of between 1 minute and 60 minutes, and applying a formulation to said area, either during heating or immediately subsequent thereto, said formulation comprising said drug and at least one permeation enhancer as described in claim 28.
